(19) United States
(12) Patent Application Publication
(10) Pub. No.: US 2015/0232944 A1
(43) Pub. Date: Aug. 20, 2015

(54) METHOD FOR PROGNOSIS OF GLOBAL SURVIVAL AND SURVIVAL WITHOUT RELAPSE IN HEPATOCELLULAR CARCINOMA

(71) Applicants: INTEGRAGEN, Evry (FR); INSTITUT NATIONAL DE LA SANTE ET DE LA RECHERCHE MEDICALE (INSERM), Paris (FR); UNIVERSITE PARIS DESCARTES, Paris (FR)

(72) Inventors: Aurelien De Reynies, Boulogne-Billancourt (FR); Pierre Laurent-Puig, Meudon (FR); Jessica Zucman-Rossi, Paris (FR); Jean-Charles Nault, Paris (FR)

(73) Assignee: Integragen, Evry (FR)

(21) Appl. No.: 14/429,515

(22) PCT Filed: Sep. 23, 2013
(86) PCT No.: PCT/EP2013/069753

§ 371 Date: Mar. 19, 2015

Related U.S. Application Data
(60) Provisional application No. 61/704,360, filed on Sep. 21, 2012.

(30) Foreign Application Priority Data
Sep. 21, 2012 (EP) 12306146.7

(51) Int. Cl. C12Q 1/68; G06F 19/20; G06F 19/00

(52) U.S. Cl. CPC C12Q 1/6886; G06F 19/345; G06F 19/20; C12Q 2600/16; C12Q 2600/158; C12Q 2600/118

(57) ABSTRACT

The present invention relates to the technical field of hepatocellular carcinoma (HCC) management, and more precisely to the prognosis of HCC aggressiveness and associated therapeutic decisions. The invention provides a new prognosis method of HCC aggressiveness, based on determination in vitro and analysis of an expression profile comprising genes TAF9, RAMP3, HN1, KRT19, and RAN. The invention also provides kits for the prognosis of HCC aggressiveness, and methods of treatment of HCC in a subject based on a preliminary prognosis of said subject HCC aggressiveness.

### TECHNICAL FIELD OF THE INVENTION

The present invention relates to the technical field of hepatocellular carcinoma (HCC) management, and more precisely to the prognosis of HCC aggressiveness and associated therapeutic decisions. The invention provides a new prognosis method of HCC aggressiveness, based on determination in vitro and analysis of an expression profile comprising genes TAF9, RAMP3, HN1, KRT19, and RAN. The invention also provides kits for the prognosis of HCC aggressiveness, and methods of treatment of HCC in a subject based on a preliminary prognosis of said subject HCC aggressiveness.

### BACKGROUND ART

Hepatocellular tumors are composed of a heterogeneous group of tumors, including malignant (hepatocellular carcinoma or HCC) and benign (hepatocellular adenoma or HCA, focal nodular hyperplasia or FNH, and regenerative macronodule) tumors.

HCC constitutes a major health problem in Asia and Africa, mainly explain by the high rate of chronic hepatitis B infection, but it incidence also rises constantly in western countries, where more than 90% of HCC develop on cirrhosis. In Western countries, the main causes of the underlining liver disease are chronic hepatitis B and C and alcohol consumption. Non-alcoholic steato-hepatitis, as a consequence of metabolic syndrome, is also an increasing cause of chronic liver disease and HCC. More rarely (around 10% of cases) HCC develops on a non-cirrhotic liver.

Surgical resection represents an important curative treatment of HCC but is impaired by a high rate of recurrence (50% to 70% at 5 years) and tumor related death (30% to 50% at 5 years) (Ishizawa T Gastroenterology 2008).

There is thus a need for simple tools permitting to predict or prognose HCC patients' overall survival and early tumor recurrence.

Indeed, depending on the aggressiveness of the HCC of the patient, said patient's clinical management should be different:
In case of low aggressiveness (i.e. good prognosis of overall survival and early recurrence), follow up only could be recommended;
In contrast, in case of high aggressiveness (i.e. bad prognosis of overall survival and early recurrence), adjuvant treatment using cytotoxic chemotherapy (doxorubicin or association of gemcitabine and oxaliplatine) or targeted therapy (sorafenib) could be recommended.

In this setting, a simple prognosis tool based on molecular profiling of a subject's liver sample would be very helpful.

Some genes such as EPCAM (Yamashita T, et al. 2008; Lee J S, et al. 2006) and KRT19 (Lee J S, et al. 2006; Durnez A, et al, 2006) have been associated to HCC prognosis.

Early recurrence, defined by tumor recurrence within the 2 years following surgery, is mainly related to tumor biology (Imamura H J hepatol 2003). The inventors have previously described a molecular classification of HCC into 6 subgroups (G1-G6) and have showed that HCC of the G3 subgroup have a poor prognosis (Boyault S Hepatology 2007; Villanueva A Gastroenterology 2011; WO2007/063118A1). Other molecular signatures of HCC recurrence and related death have been published but few of them have been externally validated (Villanueva A, clinical cancer res 2010). One of the validated molecular prognostic classifications was the G3-signature that has been previously validated in paraffin-embedded tissues (Boyault S Hepatology 2007, Villanueva A, gastroenterology 2011). In addition, several signatures for prognosis of survival without relapse (a good prognosis being associated to no relapse during the first 4 post-operative years; a bad prognosis being associated to relapse during the first 2 post-operative years) have also been described in WO2007/063118A1.

In contrast, late recurrence, defined by tumor recurrence 3 years or more after surgery, is mainly related to the feature of the surrounding non-tumor tissue (“carcinogenic field effect”). A molecular signature of 196 genes derived from non-tumor liver sample is associated with late recurrence and overall survival, and can be considered as a surrogate marker of the severity and of the carcinogenic potential of the underlining cirrhosis (Hoshida Y, NEJM, 2008). In addition, several signatures for prognosis of global survival (with or without relapse) at 5 years have also been described in WO2007/063118A1.

While the above prior art tools are useful for prognosis of HCC aggressiveness, there is still a need for validated and more powerful tumor molecular signature, in order to predict overall survival and early recurrence of resected HCC.

In particular, in view of the distinct therapeutic managements selected depending on the prognosis, it is crucial that the method of prognosis used for taking this type of therapeutic decision be highly sensitive and specific, and show high positive predictive value (PPV), negative predictive value (NPV) and accuracy (as measured by the area under the ROC curve or AUC).

In addition, it would be very useful for clinicians if a unique molecular signature was able to predict both overall survival and early recurrence. In this respect, we note that prognosis tools described in the prior art are always different for prognosis overall survival and early recurrence. Notably, best predictors of global survival (i.e. overall survival) and of survival without relapse (which also predicts early recurrence) disclosed in WO2007/063118A1 are different, which is not practical for clinicians.

In addition, many studies trying to identify molecular signature of HCC prognosis are based on cohorts of patients with specific etiologies (such as HBV- or HCV-related HCC, see Nault J C, semliver dis 2011, Woo H G gastroenterology 2011, Hsu H C Am J pathol 2000), and the general applicability of molecular signatures identified on such cohorts may be questioned and in any case needs further validation in patients with other HCC etiology.

There is thus still a need for a simple and highly reliable prognosis tool, which would permit to predict both overall survival and early recurrence and would show high sensitivity, specificity, PPV, NPV and accuracy.

Based on a new strategy of analysis of microarray data obtained from various HCC samples, the inventors have constructed a simple and reliable molecular prognosis tool that fulfills the above criteria:
It is very simple to use, since it permits to simultaneously predict overall survival and early recurrence. In addition, the analysis of the expression levels of only 5 genes is necessary for the prognosis, which also contributes to the simplicity of the test; and
It is highly reliable, since the time-dependent area under the curve (AUC) to predict tumor related death reached 0.80 in the validation cohort of 119 patients. The high number of patients included, as well as the various etiologies of their HCC cancers, further guarantees the reliability and general applicability of the test. In particular, the prognosis is independent of cirrhotic ground, tumor size and pathological features.

### DESCRIPTION OF THE INVENTION

The present invention thus relates to a method of in vitro prognosis of global survival and/or survival without relapse in a subject suffering from HCC from a liver sample of said subject, comprising:
a) Determining in vitro from said liver sample an expression profile comprising or consisting of the 5 following genes: TAF9, RAMP3, HN1, KRT19, and RAN, and optionally one or more internal control genes, or an Equivalent Expression Profile thereof; and
b) Prognosing global survival and/or survival without relapse based on said expression profile, using an algorithm calibrated with at least one reference HCC liver sample.

By “subject”, it is meant any human subject, regardless of sex or age. The subject is affected with HCC, and has preferably been subjected to a surgical liver tumor resection.

According to the invention, a “prognosis” of HCC evolution means a prediction of the future evolution of a particular HCC tumor relative to the patient suffering of this particular HCC tumor. The method according to the invention allows simultaneously for both a global survival prognosis and a survival without relapse prognosis.

By “global survival prognosis” is meant prognosis of survival, with or without relapse. As stated before, the main current treatment against HCC is tumor surgical resection. As a result, a “bad global survival prognosis” is defined as the occurrence of death within the 3 years after liver resection, whereas a “good global survival prognosis” is defined as the lack of death during the 5 post-operative years.

By “survival without relapse prognosis” is meant prognosis of survival in the absence of any relapse or recurrence. A “bad survival without relapse prognosis” is defined as the presence of tumor-relapse within the two years after liver resection, whereas a “good survival without relapse prognosis” is defined as the lack of relapse during the 4 post-operative years. By “relapse” or “recurrence”, it is meant the growing back of HCC in the same subject, after initial treatment, generally by tumor surgical resection.

In the above methods according to the invention, reference samples are used in order to calibrate an algorithm, which may then be used to prognose global survival and/or survival without relapse. In advantageous embodiments of the methods of the invention, reference samples used for calibrating the algorithm(s) used for prognosing global survival and survival without relapse are the following:
a) For prognosing global survival: at least one (preferably several) HCC sample from a patient that survived at least 5 years after tumor resection and at least one (preferably several) HCC sample from a patient that died within 3 years after tumor resection;
b) For prognosing survival without relapse: at least one (preferably several) HCC sample from a patient that did not relapse during at least 4 years after tumor resection and at least one (preferably several) HCC sample from a patient that relapsed within 2 years after tumor resection.

In the methods according to the invention, liver samples are analyzed. By “liver sample”, it is meant any sample obtained by taking part of the liver of a subject. By “HCC liver sample”, it is meant a liver sample from a subject affected with HCC. Such liver samples may notably be a liver biopsy or a partial or whole liver tumor surgical resection. Reference samples used for calibrating the algorithm are also liver samples, preferably of the same type as those analyzed.

The above methods according to the invention are based on the in vitro determination of a particular expression profile comprising or consisting of 5 specific genes. Information concerning those 5 genes is provided in Table 1 below:

TABLE 1
Description of the 5 genes included in the prognosis method of the invention,
as well as genes considered as equivalents, i.e. the at most 10 genes which
expression in HCC samples is best correlated to the original gene, with a Pearson's
correlation coefficient ≧0.3 or ≦−0.3.

| | | | | Equivalent genes |
|---|---|---|---|---|
| | | | | among the 103 genes |
| Gene short | | Chromosome | | tested in quantitative |
| name | HUGO Gene name | location | Biological functions | PCR (see legend) |
| HN1 | Hematological and | 17q25.1 | Regulation of androgen | AURKA; BIRC5; |
| | neurological | | receptor | CCNB1; CDC20; |
| | expressed 1 | | | ENO1; G6PD; GLA; |
| | | | | HSPA4; KPNA2; |
| | | | | NRAS; PDCD2; RAN; |
| | | | | SAE1; TRIP13; |
| | | | | CKS2; RRM2; |
| | | | | DLGAP5 |
| KRT19 | Keratin 19 | 17q21-q23 | Structural integrity of | CYP2C9; GNMT; |
| | | | epithelial cells, liver stem | HN1; IGF2BP3; |
| | | | cell marker | NPEPPS; NTS; |
| | | | | RARRES2; TBX3; |
| | | | | C8A; EPCAM; |
| | | | | AKR1C1.AKR1C2 |
| RAMP3 | Receptor (G protein- | 7p13-p12 | Adrenomedullin receptor, | ANGPT1; BIRC5; |
| | coupled) | | vasodilatation, | CCL5; CCNB1; |
| | activity modifying protein 3 | | angiogenesis | CYP2C9; ESR1; |
| | | | | GIMAP5; GNMT; |
| | | | | HAMP; KLRB1; |
| | | | | LCAT; SDS; UGT2B7; |
| | | | | CKS2; STEAP3; |
| | | | | RRM2; CYP2C19; |
| | | | | C8A |
| RAN | RAN, member RAS | 12q24.3 | Ras/raf pathway, control | C14orf156; CCNB1; |
| | oncogene family | | of | DPP8; ENO1; G6PD; |
| | | | DNA synthesis and cell | GLA; HN1; HSPA4; |
| | | | cycle progression | KPNA2; NRAS; |
| | | | | PDCD2; PSMD1; |
| | | | | SAE1; TAF9 |
| TAF9 | TAF9 RNA polymerase II, | 5q11.2-q13.1 | transcriptional activation, | ARFGEF2; CCNB1; |
| | TATA box binding protein | | gene regulation | DPP8; HSPA4; |
| | (TBP)-associated | | associated with apoptosis | KPNA2; NRAS; RAN; |
| | factor, 32 kDa | | | SAE1 |

In the above method according to the invention, prognosis of global survival and/or survival without relapse is made based on an expression profile comprising or consisting of 5 specific genes, and optionally one or more internal control genes, or Equivalent Expression Profiles thereof. By “expression profile”, it is meant the expression levels of the group of genes included in the expression profile. By “comprising”, it is intended to mean that the expression profile may further comprise other genes. In contrast, by “consisting of”, it is intended to mean that no further gene is present in the expression profile analyzed. By “Equivalent Expression Profile thereof” or “EEP”, it is intended to mean the original expression profile (to which said EEP is equivalent), wherein the addition, deletion or substitution of some of the genes (preferably at most 1 or 2 genes) does not change significantly the reliability of the diagnosis.

In a preferred embodiment, Equivalent Expression Profiles include expression profiles in which one of the genes of a selected genes combination is replaced by an equivalent gene. In the present description, a first gene (“gene A”) can be considered as equivalent to another second gene (“gene B”), when replacing “gene A” in the expression profile of by “gene B” does not significantly impact the performance of the test. This is typically the case when “gene A” is correlated to “gene B”, meaning that the expression of “gene A” is statistically correlated to the expression level of “gene B”, as determined by a measure such as Pearson's correlation coefficient. The correlation may be positive (meaning that when “gene A” is upregulated in a patient, then “gene” B is also upregulated in that same patient) or negative (meaning that when “gene A” is upregulated in a patient, then “gene B” is downregulated in that same patient). A maximum of 10 genes among the 103 genes analyzed by the inventors using quantitative PCR, which are the best correlated to each of the 5 genes necessary for prognosis, and which have an average Pearson's correlation coefficient ≧0.3 or ≦−0.3 are mentioned in Table 1 above.

By “determining an expression profile”, it is meant the measure of the expression level of a group a selected genes. The expression level of each gene may be determined in vitro either at the proteic or at the nucleic level, using any technology known in the art. For instance, at the proteic level, the in vitro measure of the expression level of a particular protein may be performed by any dosage method known by a person skilled in the art, including but not limited to ELISA or mass spectrometry analysis. These technologies are easily adapted to any liver sample. Indeed, proteins of the liver sample may be extracted using various technologies well known to those skilled in the art for ELISA or mass spectrometry in solution measure. Alternatively, the expression level of a protein in a liver sample may be analyzed using mass spectrometry directly on the tissue slice.

In a preferred embodiment of a method according to the invention, the expression profile is determined in vitro at the nucleic level. At the nucleic level, the in vitro measure of the expression level of a gene may be carried out either directly on messenger RNA (mRNA), or on retrotranscribed complementary DNA (cDNA). Any method to measure the expression level may be used, including but not limited to microarray analysis, quantitative PCR, southern analysis.

In a preferred embodiment of a method according to the invention the expression profile is determined in vitro using a nucleic acid microarray, in particular an oligonucleotide microarray. In another preferred embodiment of a method according to the invention, the expression profile is determined in vitro using quantitative PCR. In any case, the expression level of any gene is preferably normalized. There are many methods for normalizing obtained expression data, depending on the technology used for measuring expression. Such methods are well known to those skilled in the art. In some embodiments, normalization may be performed in comparison to the expression level of an internal control gene, generally a household gene, including but not limited to ribosomal RNA (such as for instance 18S ribosomal RNA) or genes such as HPRT1 (hypoxanthine phosphoribosyltransferase 1), UBC (ubiquitin C), YWHAZ (tyrosine 3-monooxygenase/tryptophan 5-monooxygenase activation protein, zeta polypeptide), B2M (beta-2-microglobulin), GAPDH (glyceraldehyde-3-phosphate dehydrogenase), FPGS (folylpolyglutamate synthase), DECR1 (2,4-dienoyl CoA reductase 1, mitochondrial), PPIB (peptidylprolyl isomerase B (cyclophilin B)), ACTB (actin β), PSMB2 (proteasome (prosome, macropain) subunit, beta type, 2), GPS1 (G protein pathway suppressor 1), CANX (calnexin), NACA (nascent polypeptide-associated complex alpha subunit), TAX1BP1 (Taxi (human T-cell leukemia virus type I) binding protein 1), and PSMD2 (proteasome (prosome, macropain) 26S subunit, non-ATPase, 2).

In the context of the present invention, “expression values” (also referred to as “expression levels”) of genes used for the prognosis include both:
non-normalized raw expression values, and
derivatives of raw expression values, which may further have been normalized no matter with method is used for normalization.
In particular, when quantitative PCR is used for measuring in vitro expression values of genes used for prognosis, derivatives of raw expression values selected from ΔCt, −ΔCt, ΔΔCt, or −ΔΔCt values may be used.
When a microarray is used for measuring in vitro expression values of genes used for prognosis, log derivatives (in particular log 2 derivatives) of raw expression values (which may further have been normalized or not) are usually used.

These technologies are also easily adapted to any liver sample. Indeed, several well-known technologies are available to those skilled in the art for extracting mRNA from a tissue sample and retrotranscribing mRNA into cDNA.

Many algorithms may be used for prognosing global survival and/or survival without relapse based on the expression profile determined in vitro. In particular, the algorithm may be selected from PLS (Partial Least Square) regression, Support Vector Machines (SVM), linear regression or derivatives thereof (such as the generalized linear model abbreviated as GLM, including logistic regression), Linear Discriminant Analysis (LDA, including Diagonal Linear Discriminant Analysis (DLDA)), Diagonal quadratic discriminant analysis (DQDA), Random Forests, k-NN (Nearest Neighbour) or PAM (Predictive Analysis of Microarrays) algorithms. Cox models may also be used. Centroid models using various types of distances may also be used.

A group of reference samples, which is generally referred to as training data, is used to select an optimal statistical algorithm that best separates good from bad prognosis (like a decision rule). The best separation is usually the one that misclassifies as few samples as possible and that has the best chance to perform comparably well on a different dataset.

For a binary outcome such as good/bad prognosis, linear regression or a generalized linear model (abbreviated as GLM), including logistic regression, may be used.

Linear regression is based on the determination of a linear regression function, which general formula may be represented as:

ƒ(x₁, . . . ,x_{N})=β₀+β₁x₁+ . . . +β_{N}x_{N}.

Other representations of linear regression functions may be used (see below). Logistic regression is based on the determination of a logistic regression function:

$$f ( z ) = \frac{{e}^{z}}{{e}^{z} + 1} = \frac{1}{1 + {e}^{- z}} ,$$

in which z is usually defined as

z==β₀+β₁x₁+ . . . +β_{N}x_{N}.

In the above linear or logistic regression functions, x₁ to x_{N} are the expression values (or derivatives thereof such as ΔCt, −ΔCt, ΔΔCt, or −ΔΔCt for quantitative PCR or logged values for microarray) of the N genes in the signature, β₀ is the intercept, and β₁ to β_{N} are the regression coefficients.

The values of the intercept and of the regression coefficients are determined based on a group of reference samples (“training data”). The value of the linear or logistic regression function then defines the probability that a test expression profile has a good or bad prognosis (when defining the linear or logistic regression function based on training data, the user decides if the probability is a probability of good or bad prognosis). A test expression profile is then classified as having a good or bad prognosis depending if the probability that it has good or bad prognosis is inferior or superior to a particular threshold value, which is also determined based on training data. Sometimes, two threshold values are used, defining an undetermined area. Other types of generalized linear models than logistic regression may also be used.

Alternative methods such as nearest neighbour (abbreviated as k-NN) are also commonly used for a new sample, based on whether the sample is closer to the group of good prognosis or to the group of bad prognosis. The notion of “closer” is based on a choice of distance (metric, such as but not limited to Euclidian distance) in the n-dimension space defined by a signature consisting of N genes useful for prognosis (thus excluding potential housekeeping genes used for normalization purpose). The distances between a test expression profile and all reference good or bad prognosis expression profiles are calculated and the sample is classified by analysis of the k closest reference samples (k being an positive integer of at least 1 and most commonly 3 or 5), a rule of classification being pre-established depending of the number of good or bad prognosis reference expression profiles among the k closest reference expression profiles. For instance, when k is 1, a test expression profile is classified as good prognosis if the closest reference expression profile is a good prognosis expression profile, and as bad prognosis if the closest reference expression profile is a bad prognosis expression profile. When k is 2, a test expression profile is classified as responding if the two closest reference expression profiles are good prognosis expression profiles, as non-responding if the two closest reference expression profiles are bad prognosis expression profiles, and undetermined if the two closest reference expression profiles include a good prognosis and a bad prognosis reference expression profile. When k is 3, a test expression profile is classified as good prognosis if at least two of the three closest reference expression profiles are good prognosis expression profiles, and as bad prognosis if at least two of the three closest reference expression profiles are bad prognosis expression profiles. More generally, when k is p, a test expression profile is classified as good prognosis if more than half of the p closest reference expression profiles are good prognosis expression profiles, and as bad prognosis if more than half of the p closest reference expression profiles are bad prognosis expression profiles. If the numbers of good prognosis and bad prognosis reference expression profiles are equal, then the test expression profile is classified as undetermined.

Other methodologies from the field of statistics, mathematics or engineering exist, for example but not limited to decision trees, Support Vector Machines (SVM), Neural Networks and Linear Discriminant Analyses (LDA). Cox models may also be used. Centroid models using various types of distances may also be used. These approaches are well known to people skilled in the art.

In summary, an algorithm (which may be selected from linear regression or derivatives thereof such as generalized linear models (GLM, including logistic regression), nearest neighbour (k-NN), decision trees, support vector machines (SVM), neural networks, linear discriminant analyses (LDA), Random forests, or Predictive Analysis of Microarrays (PAM) is calibrated based on a group of reference samples (preferably including several good prognosis reference expression profiles and several bad prognosis reference expression profiles) and then applied to the test sample. In simple terms, a patient will be classified as good prognosis (or bad prognosis) based on how all the genes in the signature compare to all the genes from a reference profile that was developed from a group of good prognosis (training data).

The notion of whether individual genes of the expression profile are increased or decreased in a good prognosis versus a bad prognosis sample is of scientific interest. For each individual gene, the gene expression levels in the good prognosis group can be compared to the bad prognosis group by the use of Student's t-test or equivalent methods. However, such binary comparisons are generally not used for prognosis when a signature comprises several distinct genes.

In an advantageous embodiment, the algorithm used for prognosing global survival and/or survival without relapse is linear regression, using the following formula:

$$Score ( sample X ) = {\sum }_{i = 1}^{N} \frac{{x}_{i} - {m}_{i}}{{w}_{i}}$$

wherein:
N represents the number of genes of the expression profile,
xᵢ, 1≦i≦N, represent the in vitro measured expression values of the N genes included in the expression profile (these values may notably correspond to ΔCt, −ΔCt, ΔΔCt, or −ΔΔCt values in quantitative RT-PCR experiments, and to logged, in particular log 2, values in microarray experiments, optionally after normalization),
mᵢ and wᵢ, 1≦i≦N, are fixed parameters calibrated with at least one reference sample, and
sample X is considered as having a good global survival and/or survival without relapse prognosis if Score(sample X) is inferior to a threshold value T, and as having a bad global survival and/or survival without relapse prognosis if Score(sample X) is superior or equal to threshold value T, wherein T has been calibrated with at least one reference sample.

In a particularly preferred embodiment, the expression profile is determined using quantitative PCR, expression values are ΔΔCt values, N is 5, threshold value T is zero, and mᵢ and 1≦i≦5, have the values displayed in following Table 2:

TABLE 2
Preferred parameters for linear regression prognosis of global
survival and/or survival without relapse after determination
in vitro of the expression profile using quantitative PCR.

| Gene | mᵢ | wᵢ |
|---|---|---|
| Gene 1 (TAF9) | −1.3354874 | −0.70319556 |
| Gene 2 (RAMP3) | −0.2179838 | 0.25587217 |
| Gene 3 (HN1) | −2.1549344 | −0.14253598 |
| Gene 4 (KRT19) | 2.2145301 | −0.05104661 |
| Gene 5 (RAN) | −1.1360639 | 0.1859979 |

The method of prognosis according to the invention as described herein may further comprise
a) Determining at least one other variable associated to prognosis, and
b) Prognosing global survival and/or survival without relapse based on the expression profile and the other variable(s), using an algorithm calibrated with at least one reference HCC liver sample.

Indeed, the inclusion of further variables independently associated to prognosis may further improve the reliability of the prognosis. Said other variables may notably be selected from G1-G6 classification (as disclosed in WO2007/063118A1, see below), BCLC (Barcelona Clinic Liver Cancer, Llovet, 1999, sem liv dis), CLIP (Cancer of the Liver Italian Program, CLIP investigators Hepatology, 1998), JIS (Japan Integrated Staging, Kudo m, J Gasterol 2003), TNM (Tumour-Node-Metastasis, AJCC cancer staging Handbook, 7^{th} ed Springer) clinical staging, Milan (Mazzaferro v, New England J Medicine 1996) and metroticket calculator (Mazzaferro v, lancet Oncol 2009) criteria, presence of cirrhosis (Hoshida y, NEJM, 2008), preoperative AFP (alpha feto protein) plasma levels (Chevret S J hepatol 1999), Edmonson grade (Edmondson Cancer, 1954), and microvascular invasion of the liver sample (Mazzaferro v, lancet Oncol 2009).

The G1-G6 classification is described below.

BCLC, CLIP, JIS, and TNM clinical stagings, Milan and metroticket calculator criteria, and Edmonson grade are well known to and easily determined by those skilled in the art of HCC diagnosis, prognosis and management for any liver sample based on common general knowledge, as described in publications mentioned above.

When other variables are determined, their values are combined with the expression profile in order to perform a global prognosis based on all variables (expression profile and further variables), using any appropriate algorithm.

In a preferred embodiment, when other variables are determined, said other variables are BCLC clinical staging and microvascular invasion of the liver sample.

In a preferred embodiment, a composite score is determined, based on the values of the other variables (in particular BCLC clinical staging and microvascular invasion) and the expression profile score, calculated as described herein.

An example of a composite score that may be used for prognosis is displayed in FIG. 5.

The present invention also relates to a kit comprising reagents for the determination of an expression profile comprising at most 65 distinct genes, wherein said expression profile comprises or consists of the following 5 genes: TAF9, RAMP3, HN1, KRT19, and RAN, and optionally one or more internal control genes, or an Equivalent Expression Profile thereof.

In a preferred embodiment, the kit according to the invention may be dedicated to the determination or one of the above mentioned expression profile, and then comprises reagents for the determination of an expression profile comprising at most 10 distinct genes, knowing that the expression profile with the highest number of genes of interest comprises 5 genes, and optionally one or more internal control gene. In another preferred embodiment, the kit according to the invention may further comprise reagents for the determination of other expression profiles of interest, which may be associated to HCC diagnosis and/or HCC classification into subgroups. In this case, the kit comprises reagents for the determination of an expression profile comprising at most 65 distinct genes, in order to be able to determine in vitro the expression levels of the additional expression profiles of interest. In particular, a classification of HCC samples into 6 subgroups G1 to G6 defined by the clinical and genetic main features displayed in following Table 3 has been described in WO2007/063118A1, which content relating to such classification is herein incorporated by reference:

TABLE 3
Definition of the 6 subgroups by the presence (+) or absence (−) of
clinical and genetic main features.

| | G1 | G2 | G3 | G4 | G5 | G6 |
|---|---|---|---|---|---|---|
| Chromosome instability | + | + | + | − | − | − |
| Early relapse and death | + | + | + | − | − | − |
| TP53 mutation | − | + | + | − | − | − |
| HBV infection | + | + | − | − | − | − |
| Low copy number | + | − | − | − | − | − |
| High copy number | − | + | − | − | − | − |
| CTNNB1 mutation | − | − | − | − | + | + |
| Satellite nodules | − | − | − | − | − | + |

This classification is based on the in vitro determination of an expression profile, which advantageously comprises or consists of the following 16 genes: RAB1A, REG3A, NRAS, RAMP3, MERTK, PIR, EPHA1, LAMAS, G0S2, HN1, PAK2, AFP, CYP2C9, CDH2, HAMP, and SAE1, and the method may notably comprise:
a) determining an expression profile comprising or consisting the 16 genes mentioned above;
b) calculating from said expression profile 6 subgroup distances; and
c) classifying said HCC tumor in the subgroup for which the subgroup distance is the lowest.

Preferably, the expression profile is determined using quantitative PCR, wherein the distance of a sample; to each subgroupₖ is calculated using the following formula:

$$Distance ( {sample}_{i} , {subgroup}_{k} ) = {\sum }_{t = 1 … 16} \frac{{( Δ Ct ( {sample}_{i} , {gene}_{t} ) - μ ( {subgroup}_{k} , {gene}_{t} ) )}^{2}}{σ ( {gene}_{t} )} ,$$

wherein for each geneₜ and subgroupₖ, the p(subgroupₖ, geneₜ) and σ(geneₜ) values are those displayed in following Table 4.

TABLE 4
Parameters for each gene and for each subgroup used in the above quantitative
PCR Distance formula

| μ | G1 | G2 | G3 | G4 | G5 | G6 | σ |
|---|---|---|---|---|---|---|---|
| gene 1 (RAB1A) | −16.39 | −16.04 | −16.29 | −17.15 | −17.33 | −16.95 | 0.23 |
| gene 2 (PAP) | −28.75 | −27.02 | −23.48 | −27.87 | −19.23 | −11.33 | 16.63 |
| gene 3 (NRAS) | −16.92 | −17.41 | −16.25 | −17.31 | −16.96 | −17.26 | 0.27 |
| gene 4 (RAMP3) | −23.54 | −23.12 | −25.34 | −22.36 | −23.09 | −23.06 | 1.23 |
| gene 5 (MERTK) | −18.72 | −18.43 | −21.24 | −18.29 | −17.03 | −16.16 | 7.23 |
| gene 6 (PIR) | −18.44 | −19.81 | −16.73 | −18.28 | −17.09 | −17.25 | 0.48 |
| gene 7 (EPHA1) | −16.68 | −16.51 | −19.89 | −17.04 | −18.70 | −21.98 | 1.57 |
| gene 8 (LAMA3) | −20.58 | −20.44 | −20.19 | −21.99 | −18.77 | −16.85 | 2.55 |
| gene 9 (G0S2) | −14.82 | −17.45 | −18.18 | −14.78 | −17.99 | −16.06 | 3.88 |
| gene 10 (HN1) | −16.92 | −17.16 | −15.91 | −17.88 | −17.72 | −17.93 | 0.54 |
| gene 11 (PAK2) | −17.86 | −16.56 | −16.99 | −18.14 | −17.92 | −17.97 | 0.58 |
| gene 12 (AFP) | −16.68 | −12.36 | −26.80 | −27.28 | −25.97 | −23.47 | 14.80 |
| gene 13 (CYP2C9) | −18.27 | −16.99 | −16.26 | −16.23 | −13.27 | −14.44 | 5.47 |
| gene 14 (CDH2) | −15.20 | −14.76 | −18.91 | −15.60 | −15.48 | −17.32 | 10.59 |
| gene 15 (HAMP) | −19.53 | −20.19 | −21.32 | −18.51 | −25.06 | −26.10 | 13.08 |
| gene 16 (SAE1) | −17.37 | −17.10 | −16.79 | −18.22 | −17.72 | −18.16 | 0.31 |

Reagents for the determination of an expression profile comprising N genes may include any reagents permitting to specifically quantify the expression levels of the genes included in said expression profile. For instance, when the expression profile is determined at the proteic level, then such reagents may include antibodies specific for each of the genes included in the expression profile. Preferably, the expression is determined at the nucleic level. In this case, reagents in the kit of the invention may notably include primers pairs (forward and reverse primers) and/or probes specific for each of the genes included in the expression profile (useful notably for quantitative PCR determination of the expression profile) or a nucleic acid microarray, in particular an oligonucleotide microarray. In the latter case, the nucleic acid microarray is a dedicated nucleic acid microarray, comprising probes for the detection of a maximum number of genes, as defined in the previous paragraph.

As indicated in background art section, the prognosis method according to the invention is important for clinicians because it will permit them, based on a unique and simple test, to assess the aggressiveness of the HCC tumor, and thus to adapt the treatment to the prognosis.

The invention thus also relates to a cytotoxic chemotherapeutic agent or a targeted therapeutic agent, for use in the treatment of HCC in a subject that has been given a bad prognosis using the prognosis method of the invention. The invention also relates to the use of a therapeutic cytotoxic chemotherapeutic agent or a targeted therapeutic agent for the preparation of a medicament intended for the treatment of HCC in a subject that has been given a bad global survival and/or survival without relapse prognosis by the prognosis method according to the invention. If the HCC of said subject has been further classified into subgroup G1 as defined above, then an IGFR1 inhibitor or an Akt/mTor inhibitor is preferred as adjuvant therapy. Alternatively, if the HCC of said subject has been further classified into subgroup G2 as defined above, then an Akt/mTor inhibitor is preferred as adjuvant therapy. Alternatively, if the HCC of said subject has been further classified into subgroup G3 as defined above, then a proteasome inhibitor is preferred as adjuvant therapy. Alternatively, if the HCC of said subject has been further classified into subgroup G5 or G6 as defined above, then a WNT inhibitor is preferred as adjuvant therapy However, current WNT inhibitors have toxicity problems, and there is still a need for more efficient and safer WNT inhibitors. By “cytotoxic chemotherapeutic agent” it is meant any suitable chemical agent useful for killing cancer cells. Cytotoxic chemotherapeutic agents currently used as adjuvant treatment of HCC and preferred in the present invention are doxorubicin, gemcitabine, oxaliplatine, and combinations thereof. Doxorubicin or association of gemcitabine and oxaliplatine are particularly preferred. By “targeted therapy”, it is intended to mean any suitable agent that selectively inhibits enzymes of a signaling pathway involved in HCC malignant transformation. Currently, Sorafenib, a small molecular inhibitor of several Tyrosine protein kinases (VEGFR and PDGFR) and Raf kinases (more avidly C-Raf than B-Raf), is approved for the adjuvant treatment of HCC is preferred in the present invention. Sorafenib is a bi-aryl urea of formula:

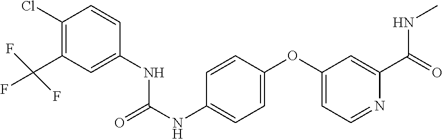 [SMILES: CNC(=O)c1cc(Oc2ccc(NC(=O)Nc3ccc(Cl)c(C(F)(F)F)c3)cc2)ccn1]

The invention also relates to a method for treating a HCC in a subject in need thereof, comprising:
a) Prognosing global survival and/or survival without relapse of said subject with the prognosis method according to the invention;
b) If said subject has been given a bad prognosis, then administering to said subject an adjuvant therapy, in particular selected from cytotoxic chemotherapy (e.g. doxorubicin or association of gemcitabine and oxaliplatine) or targeted therapy (e.g. Sorafenib).

The method of treatment of the invention may further comprise:
i. classifying said HCC sample into one of subgroups G1 to G6 using the classification method described above; and
ii. if said HCC sample is classified in G1 subgroup, then administering to said subject an efficient amount of an IGFR1 inhibitor and/or an Akt/mTor inhibitor;
iii. if said HCC sample is classified in G2 subgroup, then administering to said subject an efficient amount of an Akt/mTor inhibitor;
iv. if said HCC sample is classified in G3 subgroup, then administering to said subject an efficient amount of a proteasome inhibitor;
v. If said HCC sample is classified in G5 or G6 subgroup, then administering to said subject an efficient amount of a wnt inhibitor.

The present invention also relates to systems (and computer readable medium for causing computer systems) to perform a method of prognosis according to the invention.

In an embodiment, the invention relates to a system 1 for prognosis of global survival or survival without relapse in a subject from a liver sample of said subject, comprising:
a) a determination module 2 configured to receive a liver sample and to determine expression level information concerning an expression profile comprising or consisting of the following 5 genes: TAF9, RAMP3, HN1, KRT19, and RAN, and optionally one or more internal control genes, or an Equivalent Expression Profile thereof;
b) a storage device 3 configured to store the expression level information from the determination module;
c) a comparison module 4, adapted to compare the expression level information stored on the storage device with reference data, and to provide a comparison result, wherein the comparison result is indicative of a good or bad prognosis; and
d) optionally, a display module 5 for displaying a content 6 based in part on the classification result for the user, wherein the content is a signal indicative of a good or bad prognosis.

In another embodiment, the invention relates to a computer readable medium 7 having computer readable instructions recorded thereon to define software modules for implementing on a computer steps of a prognosis method according to the invention relating to interpretation of expression profiles data. Preferably, said software modules comprising:
a) an entry module 8, which permits expression level information relating to an expression profile comprising or consisting of the following 5 genes: TAF9, RAMP3, HN1, KRT19, and RAN, and optionally one or more internal control genes, or an Equivalent Expression Profile thereof, to be entered by a user and to be stored (at least temporarily) for further comparison;
b) a comparison module 4, adapted to compare the expression level information entered by the user with reference data and to provide a comparison result, wherein the comparison result is indicative of a good or bad prognosis; and
c) a display module 5, for displaying a content 6 based in part on the classification result for the user, wherein the content is a signal indicative of a good or bad prognosis.

Embodiments of the invention relating to systems and computer-readable media have been described through functional modules, which are defined by computer executable instructions recorded on computer readable media and which cause a computer to perform method steps when executed. The modules have been segregated by function for the sake of clarity. However, it should be understood that the modules need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable medium can be any available tangible media that can be accessed by a computer. Computer readable medium includes volatile and nonvolatile, removable and non-removable tangible media implemented in any method or technology for storage of information such as computer readable instructions, data structures, program modules or other data. Computer readable medium includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media, may define instructions, for example, as part of one or more programs, that, as a result of being executed by a computer, instruct the computer to perform one or more of the functions described herein (e.g., in relation to system 1, or computer readable medium 7), and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either system 1, or computer readable medium 6 described herein, may be distributed across one or more of such components, and may be in transition there between.

The computer-readable media may be transportable such that the instructions stored thereon can be loaded onto any computer resource to implement the aspects of the present invention discussed herein. In addition, it should be appreciated that the instructions stored on the computer readable media, or the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997, ref 38); Salzberg, Searles, Kasif, (Ed.), Computational Methods in Molecular Biology, (Elsevier, Amsterdam, 1998, ref 39); Rashidi and Buehler, Bioinformatics Basics: Application in Biological Science and Medicine (CRC Press, London, 2000, ref 40) and Ouelette and Bzevanis Bioinformatics: A Practical Guide for Analysis of Gene and Proteins (Wiley & Sons, Inc., 2^{nd} ed., 2001).

The functional modules of certain embodiments of the invention include a determination module 2, a storage device 3, a comparison module 4 and a display module 5. The functional modules can be executed on one, or multiple, computers, or by using one, or multiple, computer networks. The determination module 2 has computer executable instructions to provide expression level information in computer readable form.

As used herein, “expression level information” refers to information about expression level of any nucleotide (RNA or DNA) and/or amino acid sequences, either full-length or partial. In a preferred embodiment, it refers to the level of expression of mRNA or cDNA, measured by various technologies. The information may be qualitative (presence or absence of a transcript) or quantitative. Preferably it is quantitative.

Methods for determining expression level information, i.e. determination modules 2, include systems for protein and DNA/RNA analysis, and in particular those described above for determination of expression profiles at the nucleic or protein level.

The expression level information determined in the determination module can be read by the storage device 3. As used herein the “storage device” 3 is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices 3 also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device 3 is adapted or configured for having recorded thereon expression level information. Such information may be provided in digital form that can be transmitted and read electronically, e.g., via the Internet, on diskette, via USB (universal serial bus) or via any other suitable mode of communication including wireless communication between devices.

As used herein, “stored” refers to a process for encoding information on the storage device 3. Those skilled in the art can readily adopt any of the presently known methods for recording information on known media to generate manufactures comprising the expression level information.

A variety of software programs and formats can be used to store the expression level information on the storage device. Any number of data processor structuring formats (e.g., text file, spreadsheets or database) can be employed to obtain or create a medium having recorded thereon the expression level information.

By providing expression level information in computer-readable form, one can use the expression level information in readable form in the comparison module 4 to compare a specific expression profile with the reference data within the storage device 3. The comparison may notably be done using the various algorithms described above. The comparison made in computer-readable form provides a computer readable comparison result which can be processed by a variety of means. Content based on the comparison result can be retrieved from the comparison module 4 and displayed by the display module 5 to indicate a good or bad prognosis.

Preferably, reference data are expression level profiles that are indicative of all types of liver samples that may be found by a classification method according to the invention. The “comparison module” 4 can use a variety of available software programs and formats for the comparison operative to compare expression level information determined in the determination module 2 to reference data, either directly, or indirectly using any software providing statistical algorithms such as those already described above.

The comparison module 4, or any other module of the invention, may include an operating system (e.g., Windows, Linux, Mac OS or UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The comparison module 4 provides computer readable comparison result that can be processed in computer readable form by predefined criteria, or criteria defined by a user, to provide a content 6 based in part on the comparison result that may be stored and output as requested by a user using a display module 5. The display module 5 enables display of a content 6 based in part on the comparison result for the user, wherein the content is a signal indicative of a good or bad prognosis. Such signal can be, for example, a display of content indicative of a good or bad prognosis on a computer monitor, a printed page or printed report of content indicating a good or bad prognosis from a printer, or a light or sound indicative of a good or bad prognosis.

The content 6 based on the comparison result varies depending on the algorithm used for comparison.

For instance, when linear regression or derivatives thereof is used, the content 6 may include a score or probability of having a good or bad prognosis, or both a probability of having a good or bad prognosis and one or more threshold values, or merely a signal indicative of a good or bad prognosis. When nearest neighbor (k-NN) is used, the content 6 may include the number or proportion of good and bad prognosis expression profiles among the k closest profiles, or merely a signal indicative of a good or bad prognosis. Moreover, the content 6 may simply be a continuous or categorical score reported in a numerical, text or graphical way (for example using a color code such as red, orange or green).

The display module 5 can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or from ARM Holdings, or any other type of processor, visual display devices such as flat panel displays, cathode ray tubes and the like, as well as computer printers of various types or integrated devices such as laptops or tablets, in particular iPads.

In one embodiment, a World Wide Web browser is used for providing a user interface for display of the content 6 based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces. The requests so formulated with the user's Web browser are transmitted to a Web application which formats them to produce a query that can be employed to extract the pertinent information.

In one embodiment, the display module 5 displays the comparison result and whether the comparison result is indicative of a good or bad prognosis.

In one embodiment, the content 6 based on the comparison result that is displayed is a signal (e.g. positive or negative signal) indicative of a good or bad prognosis, thus only a positive or negative indication may be displayed.

The present invention therefore provides for systems 1 (and computer readable media 7 for causing computer systems) to perform methods of prognosing global survival and/or survival without relapse in HCC subjects, based on expression profiles information from a liver sample of said HCC subject.

System 1, and computer readable medium 7, are merely illustrative embodiments of the invention for performing methods of prognosing global survival and/or survival without relapse in HCC subjects based on expression profiles, and are not intended to limit the scope of the invention. Variations of system 1, and computer readable medium 7, are possible and are intended to fall within the scope of the invention.

The modules of the system 1 or used in the computer readable medium, may assume numerous configurations. For example, function may be provided on a single machine or distributed over multiple machines.

### DESCRIPTION OF THE FIGURES

FIG. 1. flow chart of the prognostic study.

FIG. 2. Prognosis analysis according to the 5 genes-score in training and validation cohort. Overall survival (A and B), early tumor recurrence free survival (C and D) and survival post recurrence (E) in the training and validation cohort according to the 5 genes score dichotomized in good and poor prognosis. Time-dependent AUC related to overall survival of the 5-genes score in the validation cohort (F). Subgroup analysis for overall survival among patients classified in the poor prognostic group with results expressed using Hazard ratios (G) in the whole cohort (n=314).

FIG. 3. Expression of the 5 genes included in the prognostic score. Levels of expression of the 5 genes using quantitative RT-PCR and stratified in patients with good and bad prognosis by the 5-genes score. Results were expressed in mean and normalized to normal liver tissues. Statistical analysis was performed using the non-parametric Mann-Whitney test.

FIG. 4. Overall survival in different tumor staging systems according to the 5 genes score. Subgroup analysis (HCC staging system) for overall survival was performed among patients classified in the poor prognostic group using the 5 genes score. Results were expressed using hazard ratios in the whole cohort (n=314).

FIG. 5. A composite nomogram to refine prognosis prediction. The clinico-molecular nomogram integrated the 5 genes score, BCLC classification and microvascular invasion. Each component give points and the sum of the points calculated a linear predictor and a risk of death (A). The whole population was divided in 3 subgroups according the total number of points given by the nomogram: patients at low risk (<60 points), intermediate risk (60-120 points) and high risk (>120 points) of death (B).

### EXAMPLES

### Example 1

### Identification of a Molecular Signature Permitting to Prognose Global Survival and Survival without Relapse in HCC Patients

### Patients and Methods

### Patients and Tissue Samples

Liver samples were systematically frozen following liver resection for tumor in two French University hospitals, in Bordeaux (from 1998 to 2007) and Créteil (From 2003 to 2007). A total of 550 samples were included in this work and the study was approved by the local IRB committee (CCPRB Paris Saint Louis, 1997 and 2004) and all patients gave their informed consent according to French law. Were excluded: (1) tumors with necrosis>80%, (2) tumors with RNA of poor quality or of insufficient amount, (3) HCC with non-curative resection: R1 or R2 resection or extra hepatic metastasis at the time of the surgery, (4) HCC treated by liver transplantation.

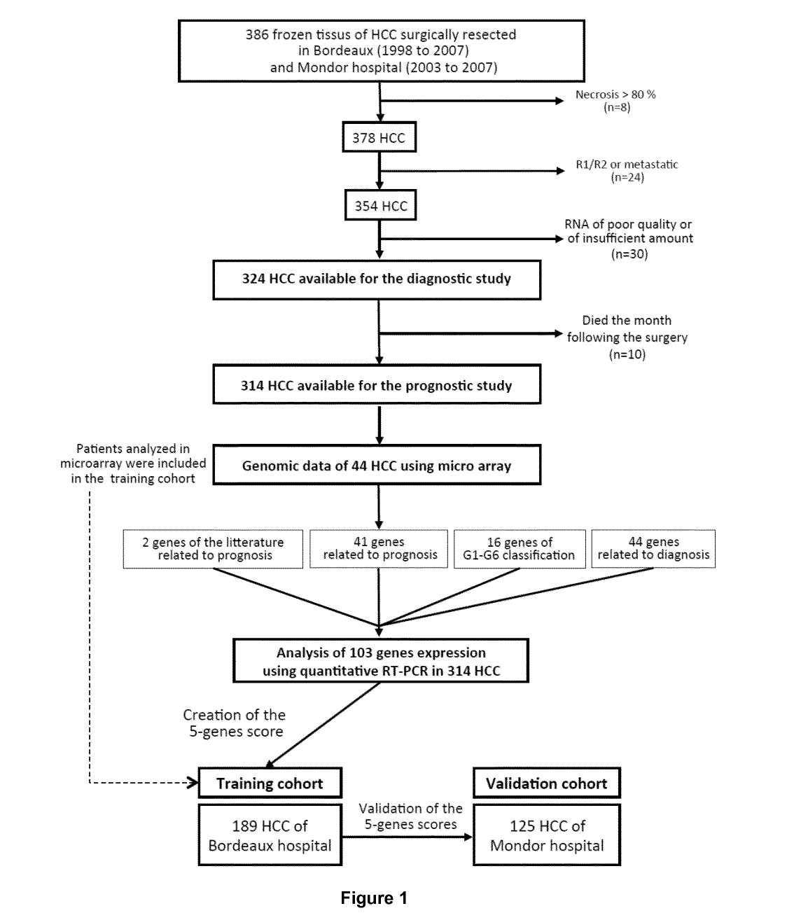

Some HCC patients (n=10) died during the month following surgery owing to surgical complications and/or decompensated cirrhosis, and were excluded from the prognostic analysis (see specific flowchart for prognosis in FIG. 1).

Accordingly, the following samples were included 324 HCC, of which 314 were qualified for the prognosis analysis, 40 non-hepatocellular tumors, 156 benign hepatocellular tumors including focal nodular hyperplasia (FNH, n=25), hepatocellular adenoma (HCA, n=111), regenerative macronodule (with dysplasia, n=15, or without, n=5) and 30 non-tumor samples.

Clinical, histological and molecular data of HCC included in prognosis analysis (n=314) are summarized in Tables 5 and 6 below:

TABLE 5
Clinical, histological and molecular data of HCC included in prognosis analysis (n = 314).

| | | | | Training | Validation | |
|---|---|---|---|---|---|---|
| | | Total | Available | cohort | cohort | |
| | Variable | n = 314 | data | n = 189 | n = 125 | P value |
| Age | >60 years* | 202 (64%) | 314 | 136 (72%) | 66 (53%) | 0.0007 |
| Gender | Male* | 252 (80%) | 314 | 156 (83%) | 96 (77%) | 0.2469 |
| Etiology | HCV* | 69 (22%) | 312 | 39 (21%) | 30 (24%) | 0.5780 |
| | HBV* | 67 (22%) | 310 | 37 (20%) | 30 (24%) | 0.4030 |
| | Alcohol* | 120 (39%) | 310 | 82 (44%) | 38 (31%) | 0.0178 |
| | NASH* | 14 (4%) | 313 | 5 (3%) | 9 (7%) | 0.0906 |
| | Hemochromatosis* | 26 (8%) | 311 | 15 (8%) | 11 (9%) | 0.8350 |
| | Miscellaneous* | 2 (1%) | 314 | 0 (0%) | 2 (2%) | 0.1577 |
| | Unknown* | 54 (17%) | 310 | 35 (19%) | 19 (15%) | 0.4471 |
| Tumor size | <5 cm* | 132 (42%) | 313 | 81 (43%) | 51 (41%) | 0.7766 |
| Tumor number | Single* | 230 (73%) | 313 | 162 (86%) | 68 (54%) | <0.0001 |
| Vascular | Microvascular* | 167 (53%) | 313 | 105 (56%) | 62 (50%) | 0.2990 |
| invasion | Macrovascular* | 44 (14%) | 313 | 26 (14%) | 18 (15%) | 0.8694 |
| Differenciation | Edmonson I-II* | 156 (51%) | 308 | 93 (51%) | 63 (50%) | 1 |
| | Edmonson III-IV* | 153 (49%) | | 91 (49%) | 62 (50%) | |
| Metavir score | F0-F1* | 117 (37%) | 315 | 73 (38%) | 44 (35%) | 0.7931 |
| (non tumor liver) | F2-F3* | 90 (29%) | | 54 (29%) | 36 (29%) | |
| | F4* | 107 (34%) | | 62 (33%) | 45 (36%) | |
| Preoperative | >20 ng/ml* | 124 (42%) | 288 | 79 (47%) | 45 (38%) | 0.1177 |
| AFP | | | | | | |
| BCLC stage | 0* | 13 (4%) | 313 | 10 (5%) | 3 (2%) | 0.0007 |
| | A* | 205 (65%) | | 134 (71%) | 71 (57%) | |
| | B* | 51 (17%) | | 18 (10%) | 33 (26%) | |
| | C* | 44 (14%) | | 26 (14%) | 18 (15%) | |
| Child Pugh | A* | 302 (97%) | 313 | 181 (96%) | 121(97%) | 0.7451 |
| | B* | 10 (3%) | | 7 (4%) | 3 (3%) | |
| 5-genes score | Good prognosis* | 177 (58%) | 306 | 96 (53%) | 81 (65%) | 0.0456 |
| | Poor prognosis* | 129 (42%) | | 85 (47%) | 44 (35%) | |
| G1-G6 | G1* | 23 (8%) | 310 | 17 (9%) | 6 (5%) | 0.0663 |
| classification | G2* | 34 (11%) | | 22 (12%) | 12 (10%) | |
| | G3* | 57 (18%) | | 32 (17%) | 25 (20%) | |
| | G4* | 80 (26%) | | 38 (20%) | 42 (34%) | |
| | G5* | 90 (29%) | | 60 (32%) | 30 (24%) | |
| | G6* | 26 (8%) | | 18 (10%) | 8 (7%) | |
| CTNNB1 | Mutated* | 100 (33%) | 307 | 61 (33%) | 39 (32%) | 0.9013 |
| TP53 | Mutated* | 62 (20%) | 303 | 36 (20%) | 26 (21%) | 0.7732 |
| Events | Median follow up | 35 (18-58) | 314 | 35 (18-55) | 35 (18-60) | 0.6803 |
| | (months)^{#} | | | | | |
| | Deaths <5 years* | 106 (34%) | 314 | 71 (38%) | 35 (28%) | 0.085 |
| | Overall recurrence | 159 (51%) | 309 | 106 (56%) | 53 (44%) | 0.0473 |
| | <5 years* | | | | | |
| | Early recurrence | 128 (41%) | 309 | 86 (45%) | 42 (35%) | 0.0760 |
| | <2 years* | | | | | |
| | Survival post | 11 (4-21) | 159 | 11 (4-20) | 9 (3-24) | 0.9431 |
| | recurrence^{#} | | | | | |

*expressed as number (%) and analyzed using fisher exact test (two-sided) except for multiple variable comparaison (chi square two sided).
^{#}expressed in months (median, 25^{th} and 75^{th} percentile) and analyzed using Mann Whitney test.

TABLE 6
Clinical classifications of HCC included in prognosis analysis (n = 314).
All variables were expressed as number (%).

| | | | | Training | Validation |
|---|---|---|---|---|---|
| | | Total | Available | cohort | cohort |
| Variable | | n = 314 | data | n = 189 | n = 125 |
| BCLC | 0 | 13 (4%) | 313 | 10 (5%) | 3 (2%) |
| classification | A | 205 (65%) | | 134 (71%) | 71 (5%7) |
| | B | 51 (17%) | | 18 (10%) | 33 (26%) |
| | C | 44 (14%) | | 26 (14%) | 18 (15%) |
| JIS | 0 | 12 (4%) | 313 | 9 (5%) | 3 (2%) |
| classification | 1 | 191 (61%) | | 128 (68%) | 63 (50%) |
| | 2 | 91 (29%) | | 45 (24%) | 46 (37%) |
| | 3 | 19 (6%) | | 6 (3%) | 13 (11%) |
| CLIP | 0 | 132 (46%) | 288 | 88 (52%) | 44 (37%) |
| classification | 1 | 112 (39%) | | 60 (36%) | 52 (44%) |
| | 2 | 38 (13%) | | 19 (11%) | 19 (16%) |
| | 3 | 6 (2%) | | 2 (1%) | 4 (3%) |
| TNM | T1 | 108 (35%) | 313 | 74 (39%) | 34 (27%) |
| classification | T2 | 126 (40%) | | 74 (39%) | 52 (42%) |
| | T3 | 79 (25%) | | 40 (22%) | 39 (31%) |
| Milan criteria | Inside | 109 (35%) | 313 | 75 (40%) | 34 (27%) |
| | Outside | 204 (65%) | | 113 (60%) | 91 (73%) |
| Metroticket | Inside | 156 (50%) | 313 | 96 (51%) | 60 (48%) |
| calculator | Outside | 157 (50%) | | 92 (49%) | 65 (52%) |
| criteria | | | | | |

Tumor and non-tumor liver samples were frozen immediately after surgery and conserved at −80° C. Tissue samples from the frozen counterpart were also fixed in 10% formaldehyde, paraffin-embedded and stained with Hematoxylin and Eosin and Masson's trichrome. The diagnosis of HCA, HCC, FNH, macroregenerative nodule and all non-hepatocellular tumors was based on established histological criteria (International working party Hepatology 1995, international consensus group Hepatology 2009). All tumors were assessed independently by 2 expert pathologists (JC and PBS) without knowledge of patient's outcome and initial diagnosis. In case of disagreement regarding the subtype diagnosis of hepatocellular tumors or regarding the pathological features of HCC included in prognosis analysis, sections were re-examined and a consensus was reached and used for the study. In the case of multitumors, the largest nodule available was analysed in our prognostic study.

### Selection of Genes for Further Analysis by Quantitative PCR

103 genes were selected for the quantitative RT-PCR analysis. Using Affymetrix HG133A gene chip TM microarray hybridizations performed on the same platform, the mRNA expression of 82 liver samples including 57 HCC (E-TABM-36), 5 HNF1A inactivated adenomas (GSE7473), 7 inflammatory adenomas (GSE11819), 4 focal nodular hyperplasia (GSE9536) 9 non-tumor liver samples including cirrhosis and normal livers (E-TABM-36 and GSE7473) was analyzed. For classification purposes, genes differentially expressed in specific subgroups of tumors were selected according to 3 criteria for inclusion:
(1) 38 genes were selected from previous microarray data obtained by the inventors and described in Boyault S, et al. 2007; Rebouissou S, et al. 2007; Rebouissou S, et al. 2009 and Rebouissou S, et al. 2008: RAB1A, REG3A, NRAS, RAMP3, MERTK, PIR, EPHA1, LAMA3, GOS2, HN1, PAK2, AFP, CYP2C9, CDH2, HAMP, SAE1, NTS, HAL, SDS, cmkOR1/CXCR7, ID2, GADD45B, CDT6, UGT2B7, LFABP, GLUL, LGR5/GPR49, TBX3, RHBG, SLPI, AMACR, SAA2, CRP, MME, DHRS2, SLC16A1, GLS2, and GNMT;
(2) 9 genes were previously described in the literature (Odom D T, et al. 2004; Paradis V, et al. 2003; Rebouissou S, et al. 2008; Llovet J, et al. 2006; Capurro M, et al. 2003; Chuma M, et al. 2003; Tsunedomi 2005; Kondoh N 1999): HNFIA, HNF4A, SERPIN, ANGPT1, ANGPT2, XLKD1-LYVE1, GPC3, HSP70/HSPA1A, and CYP3A7; and
(3) 13 genes were selected from new analysis of previous microarray data of the inventors: STEAP3, RRM2, GSN, CYP2C19, C8A, AKR1B10, ESR1, GMNN, CAP2, DPP8, LCAT, NEK7, LAPTM4B.

A total of 60 genes were selected for further analysis by quantitative PCR.

The inventors also wished to provide a new tool for simple and reliable prognosis of HCC, so that further genes found or already described as associated to HOC prognosis were also included for further quantitative PCR analysis:
(1) a panel of 41 genes mostly differentially expressed (significance and fold change) between HCC patients characterized by radically different prognosis was identified by new microarray data obtained using Affymetrix microarray E-TABM-36 analysis of the pattern of expression of 44 HCC treated by curative resection: TAF9, NRCAM, PSMD1, ARFGEF2, SPP1, CDC20, NRAS, ENO1, RRAGD, CHKA, RAN, TRIP13, IMP-3/IGF2BP3, KLRB1, C14orf156, NPEPPS, PDCD2, PHB, KIAA0090, KPNA2, KIAA0268/UNQ6077/LOC440751, G6PD, STK6, TFRC, GLA, AKR1C1/AKR1C2, GIMAP5, ADM, CCNB1, TKT, ALPS, NUDT9, HLA-DQA1, NEU1, RARRES2, BIRC5, FLJ20273, HMGB3, MPPE1, CCL5, and DLG7; and
(2) 2 genes (KRT19 and EPCAM) described in the literature as related to HCC prognosis (Lee J S, et al. 2006, Yamashita T, et al. 2008).

A total of 43 genes were selected for their association with HCC prognosis.

### Quantitative RT-PCR

RNAs extraction and quantitative RT-PCR was performed, as previously described. Expression of the 103 selected genes was analysed in duplicate in all the 550 samples using TaqMan Microfluidic card TLDA (Applied Biosystems) gene expression assays. Gene expression was normalized with the RNA ribosomal 18S, and the level of expression of the tumor sample was compared with the mean level of the corresponding gene expression in normal liver tissues, expressed as an n-fold ratio. The relative amount of RNA was calculated with the 2-delta delta CT method.

### Mutation Screening

DNA was extracted and quality was assessed. All HCA samples have been sequenced for CTNNB1 (exon 2 to 4), HNF1A (exon 1 to 10), IL6ST (exon 6 and 10), GNAS (exon 8) and STAT3 (exon 2, 5 and 20). AH HCC samples have been sequenced for CTNNB1 (exon 2 to 4) and TP53 (exons 2 to 11). All mutations were confirmed by sequencing a second independent amplification product on both strands; screening for mutations in the matched non-tumor sample was performed in order to detect any germline mutations.

### Endpoints for the Prognosis

The study design followed general recommendations of the report for markers in prognosis study REMARK (McShane L M, et al. 2005) and of EASL/EORTC guidelines (EASL J, et al. 2012). After surgery, patients were followed and HCC recurrence was screened by dosage of serum AFP and CT-SCAN (or liver MRI). The primary end point of the study was disease specific overall survival by analysing the tumor related death and we censored patients died of another etiology. Tumor related death was defined when death occurred in patients with HCC involving more than 50% of the liver, HCC with extensive tumor portal thrombosis or extrahepatic metastasis. To limit the background noise due to the occurrence of a second independent HCC, we censored survival at 5 years after the initial resection surgery. The last follow-up recorded visit was in February 2011. We also assessed survival in patients that relapse, “survival post-recurrence”, defined by the interval between tumor recurrence and death.

### Construction of the Prognosis Score

The 314 HCC were divided into a training set S1 (189 patients treated in Bordeaux) and a validation set S2 (125 patients treated in Créteil). Based on S1, univariate Cox models were calculated for each of the 103 measured genes (survival R package, coxph function, breslow method) and genes with a logrank test pvalue less than 0.05 were selected, yielding 31 genes. These 31 genes were used in a stepwise procedure with the logrank test pvalue as selection criterion, to build multivariate Cox models on S1. We used a modified stepwise forward procedure: at run k>2 (i.e. building a model at k variables, based on a previously obtained model at (k−1) variables), we add a variable, then remove a variable and add again a variable. The variable to be added or removed is selected among those optimizing the criterion. When several variables are optimizing the criterion, the first encountered is selected. We built 10 models, ranging from 1 to 10 genes. We then selected the smallest model, i.e. with the less possible variables, optimizing the criterion. To validate this model (k=5 genes), it was used to predict samples from the validation set S2.

### Prediction of Prognosis (5-Genes Dichotomized Score)

Given a sample to be classified in one of two prognostic classes 0 and 1 (respectively corresponding to favorable and pejorative outcomes), N variables and related measures X=(x1, xN) for this sample, the sample will be attributed to class 0 or 1 based on the following rule:

$$Prognosis ( X ) = {I}_{{ℝ}^{+}} ( Λ ( X ) ) , \\ \begin{matrix}i . e . Prognosis ( X ) = 1 if Λ ( X ) \geq 0 \\ 0 if Λ ( X ) < 0\end{matrix}$$

$$wherein Λ ( X ) = {\sum }_{i = 1}^{N} \frac{{x}_{i} - {m}_{i}}{{w}_{i}}$$

Parameters (mi,wi) are given in Table 2 above.

In the composite prognostic score the value of A(X) is used as an input, in addition to the BCLC class and the microvascular invasion.

### Statistical Analysis

Log rank test and Kaplan Meier method were used to assess survival. Continuous and discontinuous variable were compared using Mann Whitney and Chi square or fisher exact test respectively. Univariate and multivariate analysis were performed using the Cox model. Statistical analysis was performed using the R statistical software and rms package.

The area under the curve for testing the signature accuracy in terms of specific survival prediction was performed according to Uno, H., et al. 2007. Prediction rules were evaluated for t-year survivors with censored regression models (Journal of the American Statistical Association 102, 527-537) and using the survAUC R package. The nomogram was built by using the rms package.

### Results

### A 5-Genes Score Related to Prognosis of Resected HCC

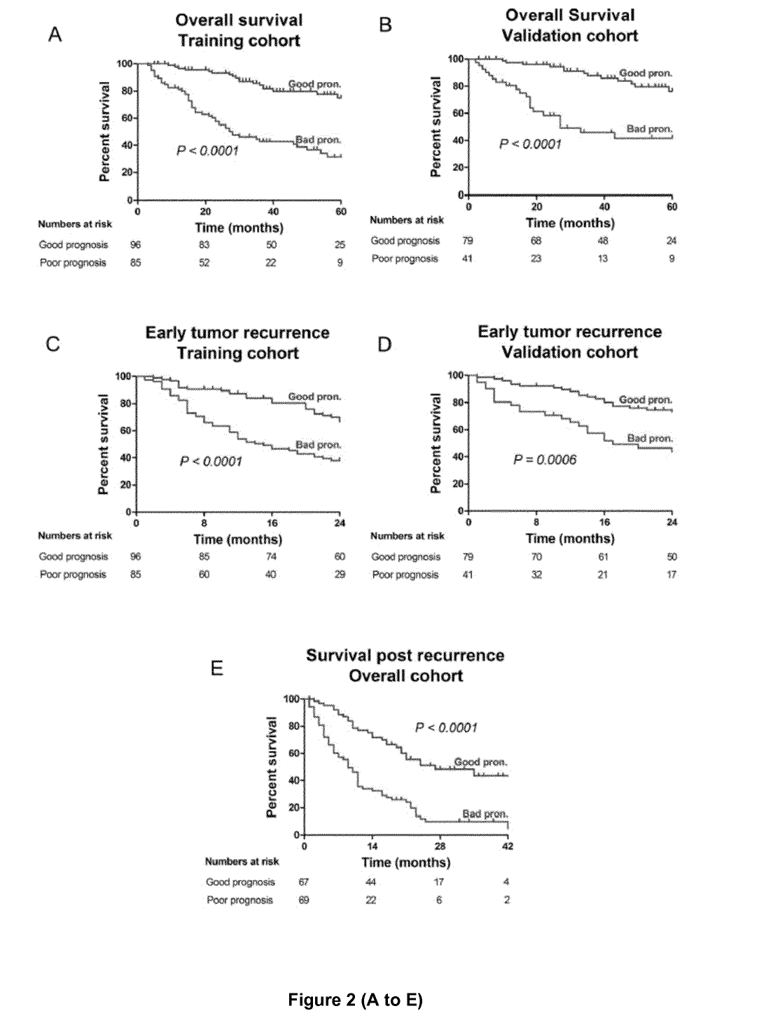

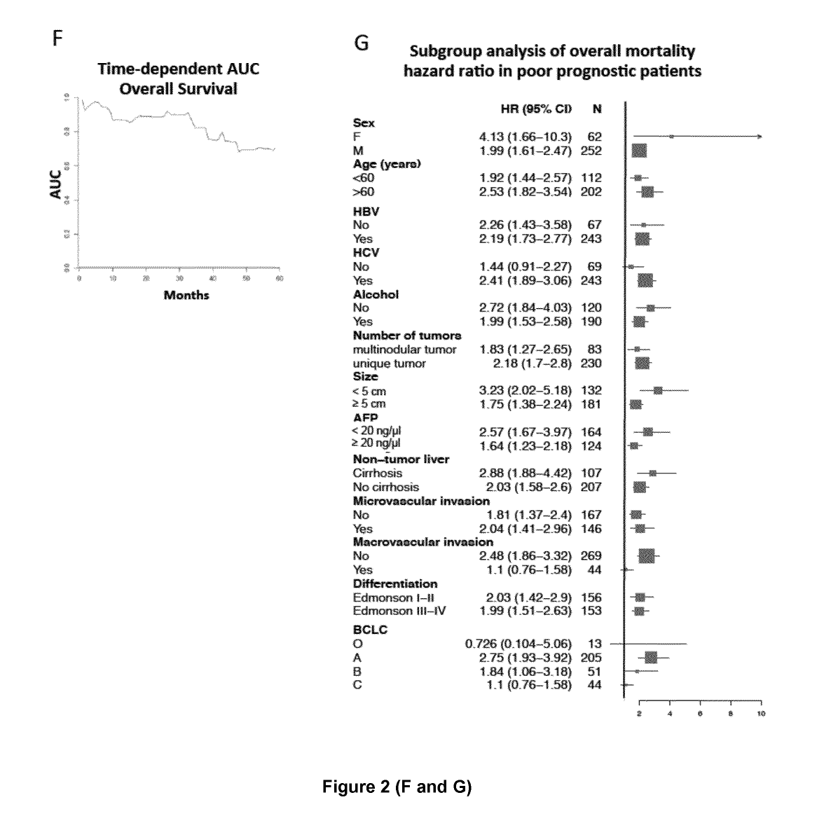

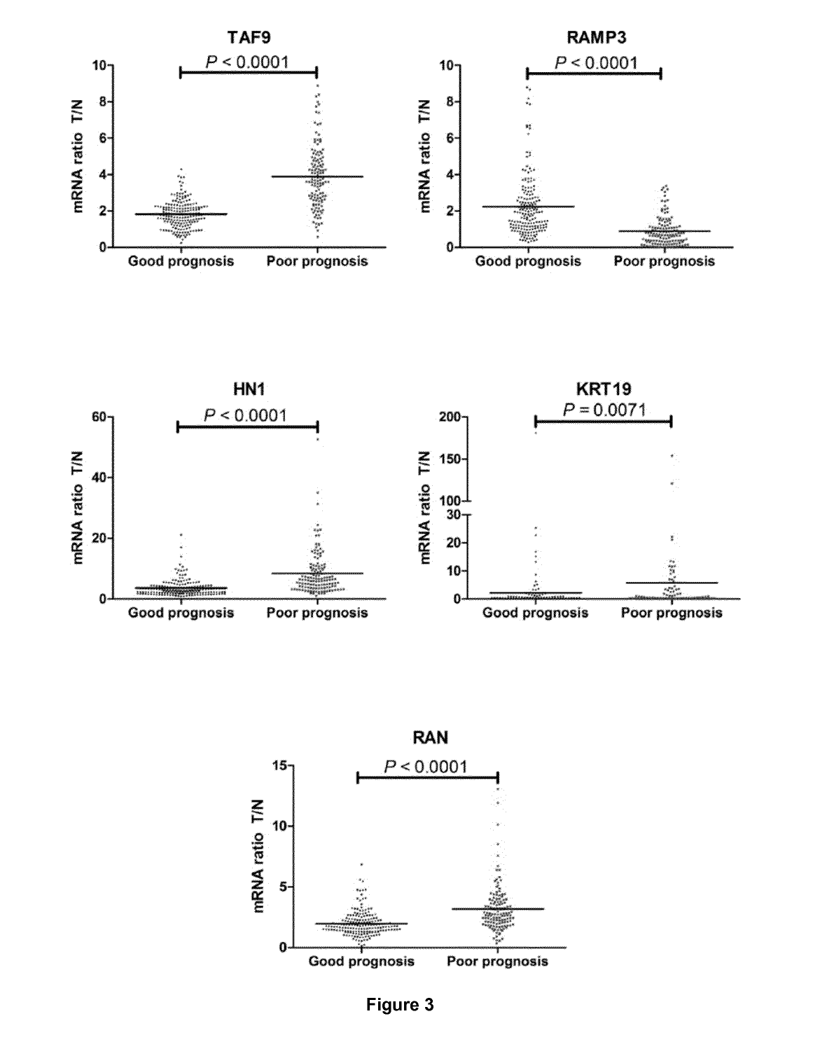

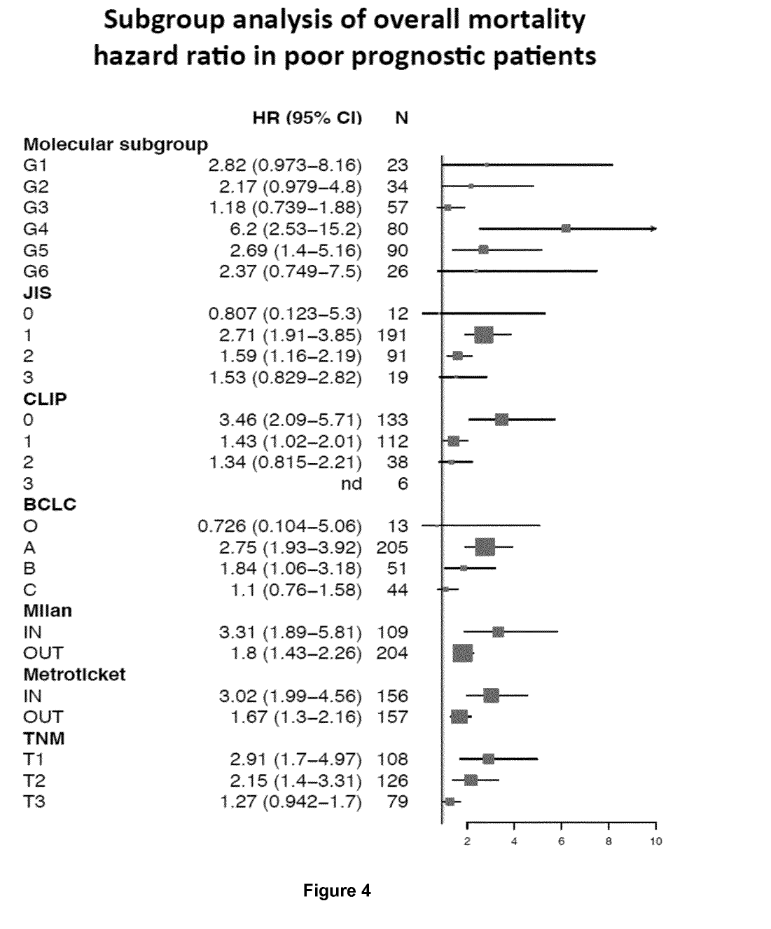

To create and validate a robust molecular genes-score to predict overall survival and early tumor recurrence of resected HCC, the expression of a set of 103 genes was analyzed in the 314 HCC qualified for prognosis (see flowchart in FIG. 1). In the training set (189 patients treated in Bordeaux), using univariate Cox analysis and a leave in-leave out strategy, a panel of 5 genes (TAF9, RAMP3, HN1, KRT19 and RAN) showing the strongest prognostic relevance was identified (see FIG. 2). Among these 5 genes, 4 were upregulated in poor prognosis HCC (see FIG. 3). Finally, a 5-genes score using the coefficient and regression formula of the multivariate Cox model was constructed from the training cohort. Then, this 5-genes score was validated in the independent validation cohort including 125 patients treated in Mondor Hospital.

The dichotomized 5-genes score was significantly associated with overall survival in the training (log rank P<0.0001, FIG. 2A) and in the validation cohort (log rank P<0.0001, FIG. 2B). To estimate the accuracy of the 5 genes score to predict overall specific survival, the AUC of the 5-genes score was calculated by building a Cox regression model on training cohort and tested on the validation cohort. The AUC was calculated for different times and is reported in FIG. 2F. The summary measure of AUC is given by the integral of AUC on 0 to 60 months and reached 0.80.

Moreover, the 5 genes score was also associated with early tumor recurrence in both the training (log rank P<0.0001, see FIG. 2C) and validation cohorts (log rank P=0.0006, see FIG. 2D).

Then, the inventors asked if the molecular prognostic classification of the primitive tumor could predict the clinical course of the corresponding relapse. Accordingly, in the subgroup of patients that relapse, the score (performed on the primitive tumor) accurately predicted the risk of death after relapse (log rank P<0.0001, see FIG. 2E). This result confirmed that patient's early relapses after surgery derive from the primitive tumor. Consequently, the 5-genes score determined by the inventors is associated with the aggressiveness of the initial tumor and relapse.

Among the 314 HCC patients treated by complete resection, 129 were classified in the poor prognosis group with the 5-genes score. This group of patients with molecular poor prognosis was significantly related to almost all the well-known clinical (HBV infection, tumor size, preoperative AFP, BCLC stage), pathological (macro and micro-vascular invasion, tumor differentiation) and molecular features (G3 classification, P53 mutations) previously associated with HCC prognosis (see Table 7 below). In contrast the molecular prognostic 5-genes score is not associated with age, other etiologies, tumor number, METAVIR score and CTNNB1 mutations.

TABLE 7
Characteristics of the patients according to the prognosis classification
with the 5-genes score (n = 306) at the time of surgery.

| | | Good | Poor | |
|---|---|---|---|---|
| | | prognosis | prognosis | |
| Variable | | n = 177 | n = 129 | P value |
| Age | >60 years* | 117 (66%) | 78 (60%) | 0.3366 |
| Gender | Male* | 144 (81%) | 101 (78%) | 0.5629 |
| Etiology | HCV* | 37 (21%) | 28 (22%) | 0.8881 |
| | HBV* | 30 (17%) | 37 (29%) | 0.0173 |
| | Alcohol* | 70 (40%) | 47 (36%) | 0.6342 |
| | NASH* | 11 (6%) | 3 (2%) | 0.1648 |
| | Hemochromatosis* | 15 (8%) | 11 (8%) | 1 |
| | Miscellaneous* | 0 (0%) | 2 (2%) | 0.1769 |
| | Unknown* | 34 (19%) | 19 (15%) | 0.3597 |
| Tumor size | <5 cm* | 87 (49%) | 43 (33%) | 0.007 |
| Tumor number | Single* | 130 (73%) | 92 (71%) | 0.6987 |
| Vascular invasion | Microvascular* | 73 (41%) | 91 (71%) | <0.0001 |
| | Macrovascular* | 12 (7%) | 31 (24%) | <0.0001 |
| Differenciation | Edmonson I-II* | 108 (61%) | 43 (33%) | <0.0001 |
| | Edmonson III-IV* | 69 (39%) | 83 (67%) | |
| Metavir score | F0-F1* | 68 (38%) | 46 (36%) | 0.3859 |
| (non tumor liver) | F2-F3* | 45 (26%) | 42 (32%) | |
| | F4* | 64 (36%) | 41 (32%) | |
| Preoperative AFP | >20 ng/ml* | 57 (35%) | 66 (56%) | 0.0004 |
| BCLC stage | 0* | 5 (3%) | 8 (6%) | <0.0001 |
| | A* | 132 (74%) | 66 (52%) | |
| | B* | 28 (16%) | 23 (18%) | |
| | C* | 12 (7%) | 31 (24%) | |
| Child pugh | A* | 171 (97%) | 123 (97%) | 1 |
| | B* | 6 (3%) | 4 (3%) | |
| G1-G6 | G1* | 10 (6%) | 13 (10%) | <0.0001 |
| classification | G2* | 12 (7%) | 22 (17%) | |
| | G3* | 8 (4%) | 48 (38%) | |
| | G4* | 69 (39%) | 7 (6%) | |
| | G5* | 63 (36%) | 27 (21%) | |
| | G6* | 14 (8%) | 10 (8%) | |
| CTNNB1 | Mutated* | 53 (31%) | 44 (35%) | 0.4548 |
| TP53 | Mutated* | 24 (14%) | 37 (30%) | 0.0012 |
| Events | Median follow up (months)^{#} | 43 (28-60) | 24 (13-42) | <0.0001 |
| | Deaths <5 years* | 30 (17%) | 73 (57%) | <0.0001 |
| | Overall recurrence <5 years* | 70 (40%) | 84 (67%) | <0.0001 |
| | Early recurrence <2 years* | 50 (29%) | 74 (59%) | <0.0001 |
| | Survival post recurrence^{#} | 17 (9-27) | 6 (2-13) | <0.0001 |

*expressed as number (%) and analyzed using fisher exact test (two-sided) except for multiple variable comparaison (chi square two sided).
^{#}expressed in months (median, 25^{th} and 75^{th} percentile) and analyzed using Mann Whitney test.

### Multivariate Analysis to Assess Prognosis of HCC Patients

The inventors also aimed to test the independent value of the new molecular 5-genes score to predict prognosis. It was showed using multivariate analysis that the 5-gene score is associated with overall survival independently of clinical and pathological features, including the BCLC staging, in the training, validation and overall cohort (see Table 8 below).

TABLE 8
Univariate and multivariate analysis of clinical, pathological and molecular
variables for overall survival in the training, validation and overall cohort.

| | UNIVARIATE ANALYSIS | | MULTIVARIATE ANALYSIS | |
|---|---|---|---|---|
| | | Wald test | | Wald test |
| Variables | HR (95% Cl) | P value | HR (95% Cl) | P value |

TRAINING COHORT (n = 189, number of death n = 71)

| | | | | |
|---|---|---|---|---|
| Gender (Male) | 1.12 (0.6-2.08) | 0.721 | | |
| Age >60 | 0.71 (0.44-1.17) | 0.182 | | |
| Etiology (HBV) | 1.03 (0.57-1.85) | 0.931 | | |
| Etiology(HCV) | 0.76 (0.45-1.3) | 0.318 | | |
| Etiology (OH) | 0.8 (0.5-1.29) | 0.36 | | |
| Cirrhosis | 1.84 (1.14-2.95) | 0.0117 | 2.33 (1.33-4.09) | 0.00313 |
| BCLC (stage B-C vs 0-A) | 3.65 (2.26-5.89) | 1.08 10⁻⁷ | 3.34 (1.85-6.01) | 5.86 10⁻⁵ |
| AFP >20 ng/ml | 1.89 (1.13-3.18) | 0.0154 | 1.49 (0.86-2.57) | 0.15 |
| No microvascular invasion | 0.33 (0.19-0.57) | 5.37 10⁻⁵ | 0.42 (0.21-0.84) | 0.0138 |
| Edmonson III/IV | 1.67 (1.03-2.71) | 0.0388 | 0.58 (0.33-1.03) | 0.0623 |
| 5-genes score (poor prognosis) | 4.67 (2.72-8.01) | 2.34 10⁻⁸ | 3.53 (1.9-6.55) | 6.24 10⁻⁵ |
| TP53 mutations | 1.33 (0.77-2.31) | 0.305 | | |
| CTNNB1 mutations | 1.31 (0.81-2.11) | 0.269 | | |

VALIDATION COHORT * (n = 125, number of death n = 35)

| | | | | |
|---|---|---|---|---|
| Gender (Male) | 5.17 (1.24-21.53) | 0.0241 | | |
| Age >60 | 0.63 (0.32-1.24) | 0.182 | | |
| Etiology (HBV) | 0.88 (0.41-1.88) | 0.743 | | |
| Etiology(HCV) | 0.87 (0.41-1.86) | 0.719 | | |
| Etiology (OH) | 0.82 (0.41-1.65) | 0.579 | | |
| Cirrhosis | 0.98 (0.49-1.96) | 0.946 | | |
| BCLC (stage B-C vs 0-A) | 3.38 (1.7-6.69) | 4.93 10⁻⁴ | 3.21 (1.53-6.76) | 0.00212 |
| AFP >20 ng/ml | 3.49 (1.71-7.09) | 5.66 10⁻⁴ | 2.2 (1.06-4.58) | 0.0354 |
| No microvascular invasion | 0.24 (0.11-0.52) | 2.73 10⁻⁴ | 0.33 (0.13-0.84) | 0.0203 |
| Edmonson III/IV | 2.06 (1.04-4.05) | 0.0373 | | |
| 5-genes score (poor prognosis) | 4.64 (2.32-9.27) | 1.37 10⁻⁵ | 2.34 (1.11-4.93) | 0.0254 |
| TP53 mutations | 1.78 (0.85-3.71) | 0.124 | | |
| CTNNB1 mutations | 1.68 (0.85-3.3) | 0.136 | | |

OVERALL (TRAINING + VALIDATION) COHORT (n = 314, number of death n = 106)

| | | | | |
|---|---|---|---|---|
| Gender (Male) | 1.72 (0.98-3.01) | 0.0601 | | |
| Age >60 | 0.74 (0.5-1.09) | 0.124 | | |
| Etiology (HBV) | 0.98 (0.62-1.56) | 0.938 | | |
| Etiology(HCV) | 0.8 (0.52-1.24) | 0.325 | | |
| Etiology (OH) | 0.78 (0.53-1.16) | 0.222 | | |
| Cirrhosis | 1.45 (0.98-2.14) | 0.0609 | 2.03 (1.3-3.18) | 0.00183 |
| BCLC (stage B-C vs 0-A) | 3.26 (2.22-4.8) | 2.02 10⁻⁹ | 2.88 (1.86-4.45) | 1.87 10⁻⁶ |
| AFP >20 ng/ml | 2.42 (1.59-3.67) | 3.34 10⁻⁵ | 1.82 (1.17-2.82) | 0.00573 |
| No microvascular invasion | 0.29 (0.19-0.45) | 3.87 10⁻⁸ | 0.42 (0.25-0.72) | 0.00779 |
| Edmonson III/IV | 1.83 (1.23-2.71) | 0.00277 | 0.81 (0.52-1.26) | 0.349 |
| 5-genes score (poor prognosis) | 4.73 (3.1-7.22) | 5.93 10⁻¹³ | 2.93 (1.84-4.66) | 5.75 10⁻⁶ |
| TP53 mutations | 1.48 (0.96-2.3) | 0.0787 | | |
| CTNNB1 mutations | 1.44 (0.98-2.13) | 0.0658 | | |

* Due to the numbers of events (35 deaths) in the validation cohort, the 4 variables most significantly associated with overall survival in univariate analysis were tested in the multivariate analysis

Interestingly, in tested patients, TP53 and CTNNB1 mutations were not related to prognosis. Moreover, while related to G3-classification (see Table 9 below), the 5-genes score was more contributive to predict prognosis in each cohort of patients (see Table 9 below).

TABLE 9
Comparison of G3 signature and the 5-genes score using bivariate
analysis in each set of patients

| | BIVARIATE ANALYSIS | |
|---|---|---|
| | | Wald test |
| Variables | HR (95% Cl) | P value |

Training cohort

| | | |
|---|---|---|
| 5-genes score (poor prognosis) | 4.66 (2.66-8.17) | 7.68 10⁻⁸ |
| G3 signature | 0.95 (0.53-1.7) | 0.868 |

Validation cohort

| | | |
|---|---|---|
| 5-genes score (poor prognosis) | 3.32 (1.43-7.73) | 0.00534 |
| G3 signature | 1.9 (0.83-4.37) | 0.131 |

Overall (training + validation) cohort

| | | |
|---|---|---|
| 5-genes score (poor prognosis) | 4.46 (2.83-7.01) | 1.04 10⁻¹⁰ |
| G3 signature | 1.16 (0.73-1.82) | 0.531 |

In addition, the performance of the 5-genes score was also compared to that of several prognosis scores disclosed in WO2007/063118A1. The 5-genes score was also found to be more contributive to predict prognosis in each cohort of patients (see Table 10 below).

TABLE 10
Comparison of the 5-genes score and of former global survival predictors
described in WO2007/063118A1 using univariate analysis in each set of patients.

| | |
|---|---|
| | Univariate analysis |
| Variables | Log rank test P value |

Training cohort (S1)

| | |
|---|---|
| 5-genes score (poor prognosis) | 8.83 10⁻¹⁰ |
| WO2007/063118A1: TAF9, NRCAM, RAMP3, PSMD1 and | 3.06 10⁻⁷ |
| ARFGEF2 signature | |
| WO2007/063118A1: TAF9, PIR, NRCAM, and RAMP3 signature | 4.30 10⁻⁷ |
| WO2007/063118A1: TAF9, NRCAM, RAMP3, and PSMD1 signature | 2.70 10⁻⁷ |
| WO2007/063118A1: TAF9, NRCAM, NRAS, RAMP3, and PSMD1 | 4.96 10⁻⁷ |
| signature | |

Validation cohort (S2)

| | |
|---|---|
| 5-genes score (poor prognosis) | 1.89 10⁻⁶ |
| WO2007/063118A1: TAF9, NRCAM, RAMP3, PSMD1 and | 5.02 10⁻³ |
| ARFGEF2 signature | |
| WO2007/063118A1: TAF9, PIR, NRCAM, and RAMP3 signature | 1.47 10⁻⁵ |
| WO2007/063118A1: TAF9, NRCAM, RAMP3, and PSMD1 signature | 1.47 10⁻³ |
| WO2007/063118A1: TAF9, NRCAM, NRAS, RAMP3, and PSMD1 | 1.47 10⁻³ |
| signature | |

Overall (training + validation) cohort (S1 + S2)

| | |
|---|---|
| 5-genes score (poor prognosis) | 2.44 10⁻¹⁵ |
| WO2007/063118A1: TAF9, NRCAM, RAMP3, PSMD1 and | 1.26 10⁻⁹ |
| ARFGEF2 signature | |
| WO2007/063118A1: TAF9, PIR, NRCAM, and RAMP3 signature | 1.14 10⁻¹¹ |
| WO2007/063118A1: TAF9, NRCAM, RAMP3, and PSMD1 signature | 3.31 10⁻¹⁰ |
| WO2007/063118A1: TAF9, NRCAM, NRAS, RAMP3, and PSMD1 | 6.06 10⁻¹⁰ |
| signature | |

As the French patients reflected the diversity of HCC in term of stages, etiologies and underlining liver diseases, the performance of the 5-genes score in each condition was analyzed (see FIG. 2G). Interestingly, the 5-genes score was significantly associated with overall survival in each subtype of HCC regardless the underlining liver disease, size of the tumor, level of tumor differentiation or the presence of micro-vascular invasion. Moreover, in patients classified by the most commonly used clinical staging, BCLC, the 5-gene score was able to refine prognosis prediction (see FIG. 2G). Similar results were obtained with 5 other clinical staging systems (CLIP, JIS, TNM classification and Milan and metroticket calculator criteria (see FIG. 3 and Table 6 above).

All these results underline the robustness and the strong independent ability of the 5-genes score to predict the prognosis of patients with HCC treated by resection.

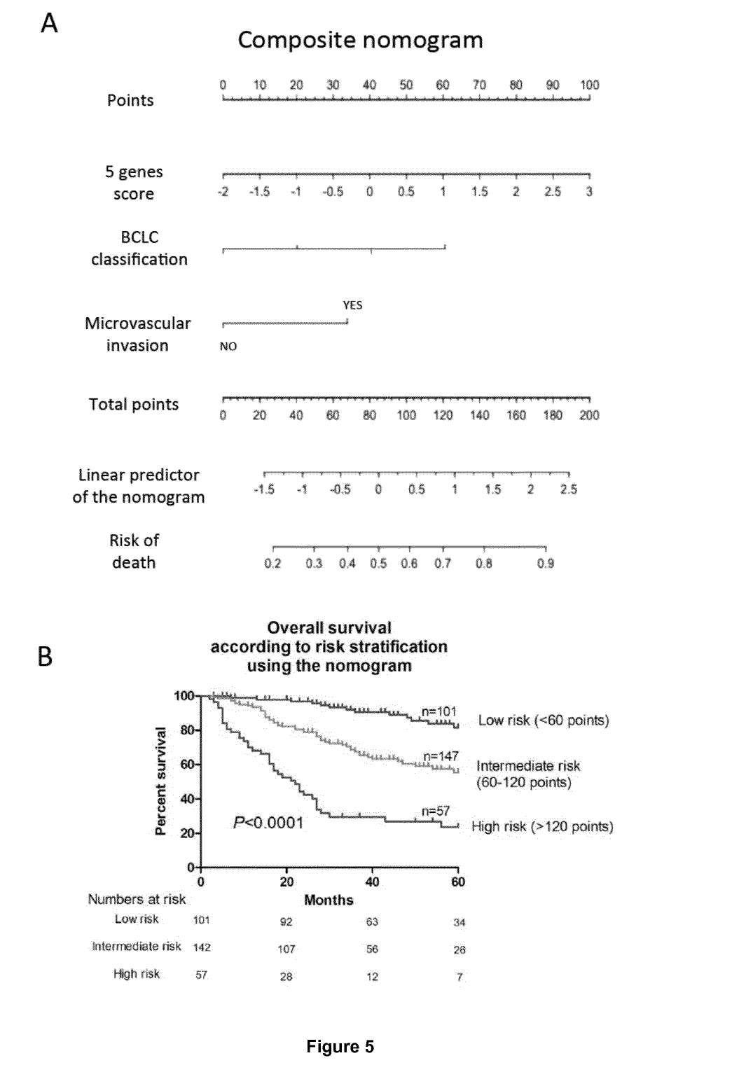

Finally, the most relevant clinical, pathological and molecular variables was assembled in the overall series of HCC patients to develop a composite prognostic predictor. Integration of the BCLC classification with microvascular invasion and the 5-genes score was performed to obtain a composite score. The nomogram in FIG. 5A shows the contribution of each variable to predict tumor-related death at 5 years. The composite scoring divided in 33^{rd} and 66^{th} percentiles accurately discriminated patients with good, intermediate and poor prognosis (see FIG. 5B).

### CONCLUSION

Molecular prediction of HCC recurrence and related death is an expanding field. More than 18 different molecular signatures have been published yet but few of them have been externally validated (Villanueva A, et al. 2010). One of these validated molecular prognostic classifications was the G3-signature that has been previously validated in paraffin-embedded tissues (Boyault S, et al. 2007, Villanueva A, et al. 2011).

The 5 genes included in the prognostic signature were TAF9, RAMP3, HN1, KRT19 and RAN. They reflected different signaling pathways deregulated in poor prognostic tumors. The stem cell/progenitor feature related to KRT19 expression was already described in poor-prognostic HCC (Lee J S nat med 2006). Similarly, TAF9, RAMP3, and HN1 had already been associated to HCC prognosis in WO2007/063118A1. In contrast, RAN is a new player in HCC prognosis. These deregulations, identified within the tumors, are related to aggressiveness of the cancer and this is linked to the early relapse after surgery and survival after relapse.

In the present work, the newly identified 5-genes score was more contributive than the G3 signature to predict the prognosis of patients with HCC treated by resection. Notably, the 5-gene signature identified most of the tumors classified in G3-subgroup (86%) as having bad prognosis, but it also identified the poor-prognosis patients with tumor classified in non-G3 molecular subgroups.

Similarly, the single newly identified 5-genes score was also found more contributive than the various signatures disclosed in WO2007/063118A1 for prognosis of global survival or survival without relapse.

In the western cohort of patients used in the present study, it was taken advantage of various etiologies (alcohol, hepatitis C and B, metabolic disease) and of various stages of the disease (from early to invasive) HCC treated similarly in two French academic hospitals. In contrast to other studies focusing mainly on HBV-related HCC (Nault J C, et al. 2011, Woo H G, et al. 2011, Hsu H C, et al. 2000), no significant association between TP53 or CTNNB1 mutations and prognosis was found. The 5-gene scoring is significantly associated with prognosis independently of tumor stage, etiology or presence of cirrhosis.

In conclusion, the 5-genes score identified by the inventors will simplify and refine the prognosis and the therapeutic decision of HCC patients.

### Example 2

### Application of the Signature Identified by Quantitative PCR to Microarray Data

The 5 genes prognosis predictor described in Example 1 is based on protocols that are designed for RT quantitative PCR ΔΔCt measurements.

10 additional versions of the same 5 genes prognosis predictor (based on an expression profile consisting of genes TAF9, RAMP3, HN1, KRT19, and RAN), dedicated to microarray data, have also been developed in order to validate the 5 genes signature.

These 10 “microarray” versions were obtained based on two distinct training sets, one based on quantitative RT-PCR data and the other on microarray data, and using 5 distinct algorithms.

More precisely, the 10 “microarray” versions were obtained as follows:
The 5 genes TAF9, RAMP3, HN1, KRT19, and RAN were mapped to Affymetrix
HG-U133A probe sets: TAF9/202168_at, RAMP3/205326_at, HN1/217755_at, KRT19/201650_at, RAN/200750_s_at.
Training set: two alternative training sets have been used:
   RT-PCR data corresponding to the training set described in Example 1 was used as a first training cohort. Expression values corresponded to ΔΔCt values; or
   46 HCCs of the E-TABM-36 dataset (http://www.ebi.ac.uk/arrayexpress/experiments/E-TABM-36) for which overall survival information and Affymetrix HG-U133A RMA normalized expression profiles were available were used as a second training cohort. In this case, values used in the predictors corresponded to log 2 derivatives of raw expression values.
Based on these two alternative training sets, 2×5 microarray versions of the prognosis predictor were obtained using the following 5 algorithms:
   Cox model using Overall Survival information with a dichotomization threshold set to 0:

$$Prognosis score ( sample X ) = {\sum }_{i = 1}^{5} \frac{{x}_{i} - {m}_{i}}{{w}_{i}}$$

wherein:
   xᵢ, 1≦i≦5, represent the in vitro measured expression values of the 5 genes included in the expression profile,
   mᵢ and wᵢ, 1≦i≦5, are the following fixed parameters:

| | 1^{st} training cohort | | 2^{nd} training cohort | |
|---|---|---|---|---|
| | (RT-PCR data) | | (microarray data) | |
| i | mi | wi | mi | wi |
| 1 (TAF9) | −1.3354874 | −0.7031956 | 8.860117 | 1.43844087 |
| 2 (RAMP3) | −0.2179838 | 0.25587217 | 7.354199 | −0.94535702 |
| 3 (HN1) | −2.1549344 | −0.142536 | 7.597593 | 1.2234661 |
| 4 (KRT19) | 2.2145301 | −0.0510466 | 4.482926 | −0.00352621 |
| 5 (RAN) | −1.1360639 | 0.1859979 | 8.982648 | −0.79278408 |

and the patient is given a good prognosis if his/her prognosis score is inferior to zero and a bad prognosis if his/her prognosis score is superior or equal to zero,
Centroïd-based using uncensored Overall Survival as variable to be predicted, and (1-Pearson coefficient of correlation) as distance, without row centering:

$$Prognosis ( sample X ) = {Arg}_{min} ( distance ( good ) ; distance ( bad ) )$$

$$wherein$$

$$distance ( good ) = 1 - \frac{1}{5} {\sum }_{i = 1}^{5} ( \frac{{x}_{i} - \overline{x}}{{σ}_{x}} ) ( \frac{{μ}_{{good}_{i}} - \overline{{μ}_{good}}}{{σ}_{{μ}_{good}}} ) and$$

$$distance ( bad ) = 1 - \frac{1}{5} {\sum }_{i = 1}^{5} ( \frac{{x}_{i} - \overline{x}}{{σ}_{x}} ) ( \frac{{μ}_{{bad}_{i}} - \overline{{μ}_{bad}}}{{σ}_{{μ}_{bad}}} )$$

wherein:
   xᵢ, 1≦i≦5, represent the in vitro measured expression values of the 5 genes included in the expression profile,
   x̅ and σₓ respectively represent the average

$$( \overline{x} = \frac{{\sum }_{i = 1}^{5} {x}_{i}}{5} )$$

and the standard deviation (σₓ=\sqrt{Σᵢ₌₁⁵(xᵢ− x̅)²}) of xᵢ values, 1≦i≦5,
μ̅_̅{̅g̅o̅o̅d̅}̅ and σ_{μ}_{_{good}} respectively represent the average

$$( \overline{{μ}_{good}} = \frac{{\sum }_{i = 1}^{5} {μ}_{{good}_{i}}}{5} )$$

and the standard deviation of μ_{good}_{ᵢ} (σ_{μ}_{_{good}}=\sqrt{Σᵢ₌₁⁵(μ_{good}_{ᵢ}− μ̅_̅{̅g̅o̅o̅d̅}̅)²}) values, 1≦i≦5,
μ̅_̅{̅b̅a̅d̅}̅ and σ_{μ}_{_{bad}} respectively represent the average

$$( \overline{{μ}_{bad}} = \frac{{\sum }_{i = 1}^{5} {μ}_{{bad}_{i}}}{5} )$$

and the standard deviation (σ_{μ}_{_{bad}}=\sqrt{Σᵢ₌₁⁵(μ_{bad}_{ᵢ}− μ̅_̅{̅b̅a̅d̅}̅)²}) of μ_{bad}_{ᵢ} values, 1≦i≦5,
μ_{good}_{ᵢ} and μ_{bad}_{ᵢ} are the following fixed parameters:

| | 1st training cohort | | 2nd training cohort | |
|---|---|---|---|---|
| | (RT-PCR data) | | (microarray data) | |
| i | μ_{good}_{ᵢ} | μ_{bad}_{ᵢ} | μ_{good}_{ᵢ} | μ_{bad}_{ᵢ} |
| 1 (TAF9) | −1.1386633 | −1.6390428 | 8.555559 | 9.192362 |
| 2 (RAMP3) | −0.4853849 | 0.2667303 | 7.610904 | 7.074156 |
| 3 (HN1) | −1.991411 | −2.3530443 | 7.356473 | 7.860633 |
| 4 (KRT19) | 2.5334881 | 1.6408852 | 4.497312 | 4.467233 |
| 5 (RAN) | −1.0148545 | −1.297179 | 8.788291 | 9.194674 |

Centroïd-based using uncensored Overall Survival as variable to be predicted, and (1-Pearson coefficient of correlation) as distance, with median row centering:

$$Prognosis ( sample X ) = {Arg}_{min} ( distance ( good ) ; distance ( bad ) )$$

$$wherein$$

$$distance ( good ) = 1 - \frac{1}{5} {\sum }_{i = 1}^{5} ( \frac{{x}_{i} - \overline{x}}{{σ}_{x}} ) ( \frac{{μ}_{{good}_{i}} - \overline{{μ}_{good}}}{{σ}_{{μ}_{good}}} ) and$$

$$distance ( bad ) = 1 - \frac{1}{5} {\sum }_{i = 1}^{5} ( \frac{{x}_{i} - \overline{x}}{{σ}_{x}} ) ( \frac{{μ}_{{bad}_{i}} - \overline{{μ}_{bad}}}{{σ}_{{μ}_{bad}}} )$$

wherein:
   xᵢ, 1≦i≦5, represent the in vitro measured expression values of the 5 genes included in the expression profile,
   x̅ and σₓ respectively represent the average

$$( \overline{x} = \frac{{\sum }_{i = 1}^{5} {x}_{i}}{5} )$$

and the standard deviation (σₓ=\sqrt{Σᵢ₌₁⁵(xᵢ− x̅)²}) of xᵢ values, 1≦i≦5,
μ̅_̅{̅g̅o̅o̅d̅}̅ and σ_{μ}_{_{good}} respectively represent the average

$$( \overline{{μ}_{good}} = \frac{{\sum }_{i = 1}^{5} {μ}_{{good}_{i}}}{5} )$$

and the standard deviation of (σ_{μ}_{_{good}}=\sqrt{Σᵢ₌₁⁵(μ_{good}_{ᵢ}− μ̅_̅{̅g̅o̅o̅d̅}̅)²}) of μ_{good}_{ᵢ} values, 1≦i≦5,
μ̅_̅{̅b̅a̅d̅}̅ and σ_{μ}_{_{bad}} respectively represent the average

$$( \overline{{μ}_{bad}} = \frac{{\sum }_{i = 1}^{5} {μ}_{{bad}_{i}}}{5} )$$

and the standard deviation (σ_{μ}_{_{bad}}=\sqrt{Σᵢ₌₁⁵(μ_{bad}_{ᵢ}− μ̅_̅{̅b̅a̅d̅}̅)²}) of μ_{bad}_{ᵢ} values, 1≦i≦5,
μ_{good}_{ᵢ} and μ_{bad}_{ᵢ} are the following fixed parameters:

| | 1st training cohort | | 2nd training cohort | |
|---|---|---|---|---|
| | (RT-PCR data) | | (microarray data) | |
| i | μ_{good}_{ᵢ} | μ_{bad}_{ᵢ} | μ_{good}_{ᵢ} | μ_{bad}_{ᵢ} |
| 1 (TAF9) | 0.10986584 | −0.3905137 | −0.16707252 | 0.469731 |
| 2 (RAMP3) | −0.1881254 | 0.5639898 | 0.25124668 | −0.2855013 |
| 3 (HN1) | 0.02942182 | −0.3322115 | −0.07128225 | 0.4328778 |
| 4 (KRT19) | 0.04183309 | −0.8507698 | 0.4099826 | 0.379904 |
| 5 (RAN) | 0.09614428 | −0.1861803 | −0.11267109 | 0.2937122 |

Centroïd-based using uncensored Overall Survival as variable to be predicted, and DQDA as distance, without row centering:

$$Prognosis ( sample X ) = {Arg min}_{j ∈ \{ A , B \}} ( {∇}_{good} ( sample X ) ; {∇}_{bad} ( sample X ) )$$

wherein

$${∇}_{good} ( sample X ) = ( {\sum }_{i = 1}^{5} \frac{{( {x}_{i} - {μ}_{{good}_{i}} )}^{2}}{{v}_{{good}_{i}}} ) + {C}_{good}$$

$${∇}_{bad} ( sample X ) = ( {\sum }_{i = 1}^{5} \frac{{( {x}_{i} - {μ}_{{bad}_{i}} )}^{2}}{{v}_{{bad}_{i}}} ) + {C}_{bad}$$

wherein:
   xᵢ, 1≦i≦5, represent the in vitro measured expression values of the 5 genes included in the expression profile, and
   μ_{good}_{ᵢ}, and μ_{bad}_{ᵢ}, ν_{good}_{ᵢ} and ν_{bad}_{ᵢ} are the following fixed parameters:

| | | | | |
|---|---|---|---|---|
| i | μ_{good}_{ᵢ} | μ_{bad}_{ᵢ} | ν_{good}_{ᵢ} | ν_{bad}_{ᵢ} |

1^{st} training cohort (RT-PCR data)

| | | | | |
|---|---|---|---|---|
| 1 (TAF9) | −1.1386633 | −1.6390428 | 0.5764442 | 0.609792 |
| 2 (RAMP3) | −0.4853849 | 0.2667303 | 1.6166561 | 2.7883844 |
| 3 (HN1) | −1.991411 | −2.3530443 | 0.9875936 | 1.0443544 |
| 4 (KRT19) | 2.5334881 | 1.6408852 | 9.53942479 | 12.4737246 |
| 5 (RAN) | −1.0148545 | −1.297179 | 0.67736 | 0.6910398 |

2^{nd} training cohort (microarray data)

| | | | | |
|---|---|---|---|---|
| 1 (TAF9) | 8.555559 | 9.192362 | 0.1501967 | 0.2989976 |
| 2 (RAMP3) | 7.610904 | 7.074156 | 0.2760526 | 0.2305511 |
| 3 (HN1) | 7.356473 | 7.860633 | 0.3001276 | 0.5369335 |
| 4 (KRT19) | 4.497312 | 4.467233 | 1.03919 | 0.7997748 |
| 5 (RAN) | 8.788291 | 9.194674 | 0.2766244 | 0.4549733 |

C_{good} and C_{bad} are defined as follows:

$${C}_{Good} = ( {\sum }_{i = 1}^{N} log ( {v}_{{Good}_{i}} ) )$$

$${C}_{Bad} = ( {\sum }_{i = 1}^{N} log ( {v}_{{Bad}_{i}} ) )$$

and
Centroïd-based using uncensored Overall Survival as variable to be predicted, and DQDA as distance, with median row centering:

$$Prognosis ( sample X ) = {Arg min}_{j ∈ \{ A , B \}} ( {∇}_{good} ( sample X ) ; {∇}_{bad} ( sample X ) )$$

wherein

$${∇}_{good} ( sample X ) = ( {\sum }_{i = 1}^{5} \frac{{( {x}_{i} - {μ}_{{good}_{i}} )}^{2}}{{v}_{{good}_{i}}} ) + {C}_{good}$$

$${∇}_{bad} ( sample X ) = ( {\sum }_{i = 1}^{N 5} \frac{{( {x}_{i} - {μ}_{{bad}_{i}} )}^{2}}{{v}_{{bad}_{i}}} ) + {C}_{bad}$$

wherein:
   xᵢ, 1≦i≦5, represent the in vitro measured expression values of the 5 genes included in the expression profile, and
   μ_{good}_{ᵢ}, and μ_{bad}_{ᵢ}, ν_{good}_{ᵢ} and ν_{bad}_{ᵢ} are the following fixed parameters:

| | | | | |
|---|---|---|---|---|
| i | μ_{good}_{ᵢ} | μ_{bad}_{ᵢ} | ν_{good}_{ᵢ} | ν_{bad}_{ᵢ} |

1^{st} training cohort (RT-PCR data)

| | | | | |
|---|---|---|---|---|
| 1 (TAF9) | 0.10986584 | −0.3905137 | 0.5764442 | 0.609792 |
| 2 (RAMP3) | −0.1881254 | 0.5639898 | 1.6166561 | 2.7883844 |
| 3 (HN1) | 0.02942182 | −0.3322115 | 0.9875936 | 1.0443544 |
| 4 (KRT19) | 0.04183309 | −0.8507698 | 9.5342479 | 12.4737246 |
| 5 (RAN) | 0.09614428 | −0.1861803 | 0.67736 | 0.6910398 |

2^{nd} training cohort (microarray data)

| | | | | |
|---|---|---|---|---|
| 1 (TAF9) | −0.16707252 | 0.469731 | 0.1501967 | 0.2989976 |
| 2 (RAMP3) | 0.25124668 | −0.2855013 | 0.2760526 | 0.2305511 |
| 3 (HN1) | −0.07128225 | 0.4328778 | 0.3001276 | 0.5369335 |
| 4 (KRT19) | 0.4099826 | 0.379904 | 1.03919 | 0.7997748 |
| 5 (RAN) | −0.11267109 | 0.2937122 | 0.2766244 | 0.4549733 |

C_{good} and C_{bad} are defined as follows:

$${C}_{Good} = ( {\sum }_{i = 1}^{N} log ( {v}_{{Good}_{i}} ) )$$

$${C}_{Bad} = ( {\sum }_{i = 1}^{N} log ( {v}_{{Bad}_{i}} ) )$$

The above results indicate that predictors based on the same genes but calibrated differently, based on another training set and/or another technology for measuring expression level and/or another algorithm) lead to comparable results.

They also show that the technology used for measuring expression level in a validation group does not need to be the same at that used for the training group.

### BIBLIOGRAPHIC REFERENCES

AJCC cancer staging Handbook, 7^{th} ed Springer.
Boyault S, Rickman D S, de Reynies A, et al. Transcriptome classification of HCC is related to gene alterations and to new therapeutic targets. Hepatology 2007; 45:42-52.
Capurro M, Wanless I R, Sherman M, et al. Glypican-3: a novel serum and histochemical marker for hepatocellular carcinoma. Gastroenterology 2003; 125:89-97.
Chevret S, Trinchet J C, Mathieu D, Rached A A, Beaugrand M, Chastang C. A new prognostic classification for predicting survival in patients with hepatocellular carcinoma. Groupe d'Etude et de Traitement du Carcinome Hépatocellulaire. J Hepatol. 1999 July; 31(1):133-41.
Chuma M, Sakamoto M, Yamazaki K, et al. Expression profiling in multistage hepatocarcinogenesis: identification of HSP70 as a molecular marker of early hepatocellular carcinoma. Hepatology 2003; 37:198-207.
CLIP investigators. A new prognostic system for hepatocellular carcinoma: a retrospective study of 435 patients: the Cancer of the Liver Italian Program (CLIP) investigators. Hepatology. 1998 September; 28(3):751-5;
Durnez A, Verslype C, Nevens F, et al. The clinicopathological and prognostic relevance of cytokeratin 7 and 19 expression in hepatocellular carcinoma. A possible progenitor cell origin. Histopathology 2006; 49:138-51.
EASL-EORTC clinical practice guidelines: management of hepatocellular carcinoma. Journal of hepatology 2012; 56:908-43.
EDMONDSON H A, STEINER P E. Primary carcinoma of the liver: a study of 100 cases among 48,900 necropsies. Cancer. 1954 May; 7(3):462-503.
Hoshida Y, Villanueva A, Kobayashi M, et al. Gene expression in fixed tissues and outcome in hepatocellular carcinoma. The New England journal of medicine 2008; 359:1995-2004.
Hsu H C, Jeng Y M, Mao T L, Chu J S, Lai P L, Peng S Y. Beta-catenin mutations are associated with a subset of low-stage hepatocellular carcinoma negative for hepatitis B virus and with favorable prognosis. The American journal of pathology 2000; 157:763-70.
Imamura H, Matsuyama Y, Tanaka E, et al. Risk factors contributing to early and late phase intrahepatic recurrence of hepatocellular carcinoma after hepatectomy. Journal of hepatology 2003; 38:200-7.
Pathologic diagnosis of early hepatocellular carcinoma: a report of the international consensus group for hepatocellular neoplasia. Hepatology 2009; 49:658-64.
Ishizawa T, Hasegawa K, Aoki T, et al. Neither multiple tumors nor portal hypertension are surgical contraindications for hepatocellular carcinoma. Gastroenterology 2008; 134:1908-16.
Kondoh N, Wakatsuki T, Ryo A, et al. Identification and characterization of genes associated with human hepatocellular carcinogenesis. Cancer research 1999; 59:4990-6.
Kudo M, Chung H, Osaki Y. Prognostic staging system for hepatocellular carcinoma (CLIP score): its value and limitations, and a proposal for a new staging system, the Japan Integrated Staging Score (JIS score). J Gastroenterol. 2003; 38(3):207-15.
Lee J S, Heo J, Libbrecht L, et al. A novel prognostic subtype of human hepatocellular carcinoma derived from hepatic progenitor cells. Nature medicine 2006; 12:410-6.
Llovet J M, Brú C, Bruix J. Prognosis of hepatocellular carcinoma: the BCLC staging classification. Semin Liver Dis. 1999; 19(3):329-38.
Llovet J M, Chen Y, Wurmbach E, et al. A molecular signature to discriminate dysplastic nodules from early hepatocellular carcinoma in HCV cirrhosis. Gastroenterology 2006; 131:1758-67.
Mazzaferro V, Regalia E, Doci R, Andreola S, Pulvirenti A, Bozzetti F, Montalto F, Ammatuna M, Morabito A, Gennari L. Liver transplantation for the treatment of small hepatocellular carcinomas in patients with cirrhosis. N Engi J Med. 1996 Mar. 14; 334(11):693-9.
Mazzaferro V, Llovet J M, Miceli R, et al; Metroticket Investigator Study Group. Predicting survival after liver transplantation in patients with hepatocellular carcinoma beyond the Milan criteria: a retrospective, exploratory analysis. Lancet Oncol. 2009 January; 10(1):35-43.
McShane L M, Altman D G, Sauerbrei W, Taube S E, Gion M, Clark G M. REporting recommendations for tumor MARKer prognostic studies (REMARK). Nature clinical practice Urology 2005; 2:416-22.
Nault J C, Zucman-Rossi J. Genetics of hepatobiliary carcinogenesis. Seminars in liver disease 2011; 31:173-87.
Odom D T, Zizlsperger N, Gordon D B, et al. Control of pancreas and liver gene expression by HNF transcription factors. Science 2004; 303:1378-81.
Ouelette and Bzevanis Bioinformatics: A Practical Guide for Analysis of Gene and Proteins (Wiley & Sons, Inc., 2^{nd} ed., 2001)
Paradis V, Bieche I, Dargere D, et al. A quantitative gene expression study suggests a role for angiopoietins in focal nodular hyperplasia. Gastroenterology 2003; 124:651-9.
Rashidi and Buehler, Bioinformatics Basics: Application in Biological Science and Medicine (CRC Press, London, 2000, ref 40)
Rebouissou S, Couchy G, Libbrecht L, et al. The beta-catenin pathway is activated in focal nodular hyperplasia but not in cirrhotic FNH-like nodules. Journal of hepatology 2008; 49:61-71.
Rebouissou S, Amessou M, Couchy G, et al. Frequent in-frame somatic deletions activate gp130 in inflammatory hepatocellular tumours. Nature 2009; 457:200-4.
Rebouissou S, Imbeaud S, Balabaud C, et al. HNF1alpha inactivation promotes lipogenesis in human hepatocellular adenoma independently of SREBP-1 and carbohydrate-response element-binding protein (ChREBP) activation. The Journal of biological chemistry 2007; 282:14437-46.
Salzberg, Searles, Kasif, (Ed.), Computational Methods in Molecular Biology, (Elsevier, Amsterdam, 1998, ref 39);
Setubal and Meidanis et al., Introduction to Computational Biology Methods (PWS Publishing Company, Boston, 1997, ref 38);
Tsunedomi R, Iizuka N, Hamamoto Y, et al. Patterns of expression of cytochrome P450 genes in progression of hepatitis C virus-associated hepatocellular carcinoma. International journal of oncology 2005; 27:661-7.
Uno, Hajime; Cai, Tianxi; Tian, Lu; and Wei, L. J., “Evaluating Prediction Rules for t-Year Survivors With Censored Regression Models” (March 2006). Harvard University Biostatistics Working Paper Series. Working Paper 38.
Villanueva A, Hoshida Y, Battiston C, et al. Combining clinical, pathology, and gene expression data to predict recurrence of hepatocellular carcinoma. Gastroenterology 2011; 140:1501-12 e2.
Villanueva A, Hoshida Y, Toffanin S, et al. New strategies in hepatocellular carcinoma: genomic prognostic markers. Clinical cancer research: an official journal of the American Association for Cancer Research 2010; 16:4688-94. WO2007/063118A1
Woo H G, Wang X W, Budhu A, et al. Association of TP53 mutations with stem cell-like gene expression and survival of patients with hepatocellular carcinoma. Gastroenterology 2011; 140:1063-70.
Yamashita T, Forgues M, Wang W, et al. EpCAM and alpha-fetoprotein expression defines novel prognostic subtypes of hepatocellular carcinoma. Cancer research 2008; 68:1451-61.

1. A method of in vitro prognosis of global survival and/or survival without relapse in a subject suffering from HCC from a liver sample of said subject, comprising:
a) Determining in vitro from said liver sample an expression profile comprising the 5 following genes: TAF9, RAMP3, HN1, KRT19, and RAN; and
b) Prognosing global survival and/or survival without relapse based on said expression profile, using an algorithm calibrated with at least one reference HCC liver sample.

2. The method of claim 1, wherein the expression profile further comprises one or more internal control genes.

3. The method of claim 1, wherein reference samples used for calibrating the algorithm(s) used for prognosing global survival and survival without relapse are the following:
i) For prognosing global survival: at least one HCC sample from a patient that survived at least 5 years after tumor resection and at least one HCC sample from a patient that died within 3 years after tumor resection;
ii) For prognosing survival without relapse: at least one HCC sample from a patient that did not relapse during at least 4 years after tumor resection and at least one HCC sample from a patient that relapsed within 2 years after tumor resection.

4. The method according to claim 1, wherein said liver sample is a liver biopsy or a partial or whole liver tumor surgical resection.

5. The method according to claim 1, wherein said expression profile is determined at the nucleic level.

6. The method according to claim 5, wherein said expression profile is determined using quantitative PCR.

7. The method according to claim 1, wherein the algorithm used for prognosing global survival and/or survival without relapse is selected from PLS (Partial Least Square) regression, Support Vector Machines (SVM), linear regression or derivatives thereof (such as the generalized linear model abbreviated as GLM, including logistic regression), Linear Discriminant Analysis (LDA, including Diagonal Linear Discriminant Analysis (DLDA)), Diagonal quadratic discriminant analysis (DQDA), Random Forests, k-NN (Nearest Neighbour), and PAM (Predictive Analysis of Microarrays) algorithms.

8. The method according to claim 7, wherein the algorithm used for prognosing global survival and/or survival without relapse is linear regression, using the following formula:
$$Score ( sample X ) = {\sum }_{i = 1}^{N} \frac{{x}_{i} - {m}_{i}}{{w}_{i}}$$
wherein:
N represents the number of genes of the expression profile,
xᵢ, 1≦i≦N, represent the in vitro measured expression values of the N genes included in the expression profile,
mᵢ and wᵢ, 1≦i≦N, are fixed parameters calibrated with at least one reference sample, and
sample X is considered as having a good global survival and/or survival without relapse prognosis if Score(sample X) is inferior to a threshold value T, and as having a bad global survival and/or survival without relapse prognosis if Score(sample X) is superior to threshold value T, wherein T has been calibrated with at least one reference sample.

9. The method according to claim 8, wherein the expression profile is determined using quantitative PCR, expression values are ΔΔCt values, N is 5, threshold value T is zero and mi and wi, 1≦i≦5, have the following values:

| Gene | mᵢ | wᵢ |
|---|---|---|
| Gene 1 (TAF9) | −1.3354874 | −0.70319556 |
| Gene 2 (RAMP3) | −0.2179838 | 0.25587217 |
| Gene 3 (HN1) | −2.1549344 | −0.14253598 |
| Gene 4 (KRT19) | 2.2145301 | −0.05104661 |
| Gene 5 (RAN) | −1.1360639 | 0.1859979 |

10. The method according to claim 1, further comprising
a) Determining at least one other variable associated to prognosis, and
b) Prognosing global survival and/or survival without relapse based on the expression profile and the other variable(s), using an algorithm calibrated with at least one reference HCC liver sample.

11. The method according to claim 10, wherein said other variables are selected from G1-G6 classification, BCLC (Barcelona Clinic Liver Cancer), CLIP (Cancer of the Liver Italian Program), JIS (Japan Integrated Staging), TNM (Tumour-Node-Metastasis) clinical staging, Milan and metroticket calculator criteria, presence of cirrhosis, preoperative AFP (alpha feto protein) plasma levels, Edmonson grade, and microvascular invasion, preferably said other variables are BCLC clinical staging and microvascular invasion of the liver sample.

12. A kit comprising reagents for the determination of an expression profile comprising at most 65 distinct genes, wherein said expression profile comprises the following 5 genes: TAF9, RAMP3, HN1, KRT19, and RAN.

13. The kit according to claim 12, wherein the expression profile further comprises one or more internal control genes.

14. The kit according to claim 12, comprising:
a) specific amplification primers and/or probes, or
b) a nucleic acid microarray.

15-16. (canceled)

17. A system 1 for prognosis of global survival or survival without relapse in a subject from a liver sample of said subject, comprising:
a) a determination module 2 configured to receive a liver sample and to determine expression level information concerning an expression profile comprising the following 5 genes: TAF9, RAMPS, HN1, KRT19, and RAN;
b) a storage device 3 configured to store the expression level information from the determination module;
c) a comparison module 4, adapted to compare the expression level information stored on the storage device with reference data, and to provide a comparison result, wherein the comparison result is indicative of a good or bad prognosis; and
d) a display module 5 for displaying a content 6 based in part on the classification result for the user, wherein the content is a signal indicative of a good or bad prognosis.

18. The system according to claim 17, wherein the expression profile further comprises one or more internal control genes.

19. A computer readable medium 7 having computer readable instructions recorded thereon to define software modules for implementing on a computer steps of a prognosis method according to claim 1 relating to interpretation of expression profiles data.

20. A method for treating a HCC in a subject in need thereof, comprising:
a) Prognosing global survival and/or survival without relapse of said subject with the prognosis method according to claim 1;
b) If said subject has been given a bad prognosis, then administering to said subject an adjuvant therapy.

21. The method of claim 20, wherein said adjuvant therapy is selected from cytotoxic chemotherapy and targeted therapy.

22. The method of claim 20, wherein said adjuvant therapy is selected from doxorubicin; association of gemcitabine and oxaliplatine; and Sorafenib.
